SEQUENCE LISTING (8 sequences)

SEQ ID NO: 1 - protein, 20 aa, synthetic construct
AKAKAKAKAKAKAKANANAN

SEQ ID NO: 2 - protein, 20 aa, synthetic construct
RRAAAARRAAAARRAAAARR

SEQ ID NO: 3 - protein, 20 aa, synthetic construct
RRRRAARRRAARRRAARRRR

SEQ ID NO: 4 - protein, 32 aa, synthetic construct
ARRRAARAARRRAARAARRRAARAARRRAARA

SEQ ID NO: 5 - protein, 20 aa, synthetic construct
RRRRRRRRRRRRRRRRRRRR

SEQ ID NO: 6 - protein, 20 aa, synthetic construct
RRRRNNRRRNNRRRNNRRRR

SEQ ID NO: 7 - protein, 20 aa, synthetic construct
AKAKAKAKAKAKAKANANAN

SEQ ID NO: 8 - protein, 20 aa, synthetic construct
RRAAAARRAAAARRAAAARR

(19) United States
(12) Patent Application Publication
(10) Pub. No.: US 2026/0053944 A1
(43) Pub. Date: Feb. 26, 2026

(54) CATIONIC PEPTIDE/PROTEIN-MODIFIED EXOSOMES FOR APPLICATIONS IN DRUG DELIVERY

(71) Applicant: Northeastern University, Boston, MA (US)

(72) Inventors: Ambika Bajpayee, Cambridge, MA (US); Chenzhen Zhang, Boston, MA (US); Matthew Warren, Groton, MA (US)

(21) Appl. No.: 19/104,816

(22) PCT Filed: Aug. 21, 2023
(86) PCT No.: PCT/US2023/030686

§ 371 Date: Feb. 19, 2025

Related U.S. Application Data
(60) Provisional application No. 63/399,317, filed on Aug. 19, 2022.

(51) Int. Cl. A61K 47/69; A61K 31/7088; A61K 35/12; A61K 47/62; A61K 48/00; A61P 19/02; C12N 15/113; C12N 15/88

(52) U.S. Cl. CPC A61K 47/6901; A61K 31/7088; A61K 35/12; A61K 47/62; A61K 48/0041; A61P 19/02; C12N 15/113; C12N 15/88

(57) ABSTRACT

Disclosed are cationic polypeptide modified exosome complexes, and methods of delivery thereof, and associated methods of treatment.

### RELATED APPLICATION

This application claims the benefit of priority to U.S. Provisional Patent Application No. 63/399,317, filed Aug. 19, 2022.

### GOVERNMENT SUPPORT

This invention was made with government support under Grant Number EB028385 awarded by the National Institutes of Health. The government has certain rights in the invention.

### BACKGROUND

Osteoarthritis (OA) affects multiple joint tissues and is associated with severe pain, inflammation, and chronic cartilage degeneration. Cartilage regeneration remains a challenge due to its avascular, a-neural, dense, and lymphatics-lacking extracellular matrix (ECM) comprising of a high density of negatively charged aggrecan-glycosaminoglycans (GAGs) and collagen II that prevents chondrocyte migration to the site of degeneration thereby inhibiting its repair. Therapeutic intervention is also limited as this dense ECM hinders intra-cartilage transport of intra-articularly (IA) administered OA drugs preventing them from reaching chondrocytes in therapeutic doses. Furthermore, drugs suffer from short joint residence time (4-6 hours) due to their rapid clearance via the synovium. As such, many clinical trials evaluating efficacy of OA biologics have failed and OA remains without a treatment.

Exosomes are 40-200 nm sized cell derived vesicles that have found applications in drug delivery due to their high biocompatibility and their role in intercellular communication owing to their cell-membrane-derived lipid bilayer and the presence of cell targeting receptors. Recent work has shown that mesenchymal stem cells (MSCs) derived exosomes can enable cartilage repair as they carry a wide range of microRNAs, mRNAs and proteins (growth factors, cytokines, chemokines) that induce regenerative processes including cell migration, proliferation, differentiation and matrix synthesis. Specifically, exosomes derived from various MSC sources (bone marrow, synovial, adipose tissue) rich in microRNA such as miR-29a, miR-29b, miR-92a-3p, miR-142-5p, and miR-129-5p have shown to play a vital role in intercellular communication for cartilage development and homeostasis by promoting chondrocyte proliferation and migration to regulate levels of chondroprotective and catabolic markers.

The negative charge of exosome lipid bilayer, however, hinders its penetration and transport into the negatively charged cartilage ECM. The density of the aggrecan-GAGs increases with depth into the cartilage, thus limiting the diffusion of particles larger than 10 nm to the deep zone (DZ) of the cartilage where chondrocytes are abundantly located². Moreover, just like therapeutic drugs, IA administered exosomes can also suffer from rapid joint clearance and their biodistribution is not well-understood. Thus, exosomes in their current form are ineffective in targeting these dense, negatively charged tissues. There exists a need for methods to modify the exosome surface in order to increase their joint residence time, enable them to penetrate and bind cartilage, and reach their chondrocyte targets in tissue deep zones.

### SUMMARY OF THE INVENTION

In certain aspects, provided herein are modified exosome complexes, comprising:
(i) an exosome;
(ii) a linking moiety;
(iii) a polypeptide residue or a protein residue;
wherein the exosome comprises a lipid bilayer;
the linking moiety is linked to the lipid bilayer via non-covalent interactions; and
the protein residue or polypeptide residue is covalently linked to the linking moiety.

In certain aspects, provided herein are methods of preparing a modified exosome complex, comprising:
(a) combining a linking moiety and an exosome comprising a lipid bilayer, thereby associating the linking moiety with the lipid bilayer of the exosome via non-covalent interactions;
(b) combining a polypeptide residue or protein residue with a buffer solution to neutralize the charge of the polypeptide residue or protein residue;
(c) combining the charge-neutralized polypeptide residue or protein residue with the linking moiety associated with the lipid bilayer of the exosome, thereby forming a covalent linkage between the charge-neutralized polypeptide residue or protein residue and the linking moiety; and
(d) bringing the buffer solution to physiological pH and salinity.

In certain aspects, provided herein are method of encapsulating RNA into an exosome comprising:
(a) combining lipofectamine and a solution comprising RNA, thereby forming a first mixture;
(b) combining the first mixture and an exosome, thereby forming a second mixture; and
(c) combining the second mixture with RNase.

In certain aspects, provided herein are methods of delivering a therapeutic agent to a negatively charged tissue, comprising administering to a subject in need thereof a therapeutically effective amount of a composition;
wherein the composition comprises a modified exosome complex and a therapeutic agent.

In certain aspects, provided herein are method of treating a joint disease, comprising administering to a subject in need thereof a therapeutically effective amount of a composition;
wherein the composition comprises a modified exosome complex and a therapeutic agent.

### BRIEF DESCRIPTION OF THE DRAWINGS

FIG. 1 shows synthesis of cartilage-targeted cationic exosomes. (a) Surface modification of exosomes by anchoring DSPE-PEG linker in the lipid bilayer of exosomes. Conjugation of (b) cationic CPCs using copper-free click chemistry and (c) avidin using non-covalent interaction with biotin to the exosome surface.

FIG. 2 shows characterization of cationic exosomes. (a) Hydrodynamic diameter (nm) and zeta potential (mV) of Exo-Avidin reacting in pH 7, 8, 9, and 10. (b) TEM images of Exosome, Exo-PEG, Exo-CPC+7K, Exo-CPC+8R, Exo-CPC+14R, and Exo-Avidin using negative staining. (c) Cellular uptake of dual-labeled cationic exosomes after 2.5 h incubation with HEK293. Exosomes were labeled with ExoGlow Red (red fluorescence), and CPCs or Avidin were labeled with FITC (green fluorescence). (d) Peptide and protein modifications of Exos were confirmed using flow cytometry. Cationic Exos were dual labeled using FITC for exosomes, Cy5 for CPC+14R, and Texas Red for avidin and captured by anti-CD63 coated magnetic beads. Thermal stability of cationic exosomes after one cycle of the freeze-thaw and 48 h culture at 37° C. by adding (e) 50 mM Trehalose and (f) 250 μM Tween 20. n=4. All data are presented as mean±s.d., P values calculated by two-tailed Student's t-test (* vs Fresh, p<0.05).

FIG. 3 shows cartilage penetration and retention property of cationic exosomes. (a) Schematic of the experimental design for characterizing cartilage tissue penetration and retention. (b) Confocal images of dual labeled Exo-CPC+7K, Exo-CPC+8R, Exo-CPC+14R, and Exo-Avidin, highlighting the diffusion in healthy and 50% GAG depleted cartilage explants from superficial (SZ) to deep zone (DZ). Exosomes were labeled with ExoGlow Red (red fluorescence), and CPCs or Avidin were labeled with FITC (green fluorescence).

FIG. 4 shows chondrocyte uptake and cytocompatibility of cationic exosomes. (a) Normalized chondrocyte uptake of surface-modified exosomes (* vs. Exo; p<0.05). (b) Cytocompatibility of primary chondrocytes after treatment with cationic exosomes for 2.5 h incubation. n=6. (c) (i) Experimental design to evaluate the effect of cationic exosomes on GAG content in healthy and OA cartilage explants. Fresh bovine cartilage explants were treated with saline and 10 ng/mL IL-1α for 5 days to create healthy and OA cartilage models, respectively. Cartilage explants were treated with exosomes and cationic exosomes for another 2 days to investigate their intra-cartilage transport and cellular uptake. (ii) Cumulative GAG loss of healthy cartilage explants on days 5 and 7. (iii) Cumulative GAG loss of OA cartilage explants on days 5 and 7. All error bars are mean±s.d. Data in a, b are compared by one-way ANOVA with post-hoc Tukey's HSD test (**** vs Exo, p<0.001).

FIG. 5 shows chondrocyte targeting ability of cationic exosomes in OA cartilage. (a) Confocal images of uptaken exosome and cationic exosomes in healthy and IL-1α treated cartilage. WGA staining (red) and DAPI (blue) were used to stain the chondrocyte membrane and nucleus, respectively, within the cartilage explant. Exosomes are labeled in green. White arrows indicate the chondrocyte and chondrocyte lacunae. (b) Green channel images show the transport of native exosome and Exo-CPC+14R through the full-thickness of IL-1α treated cartilage from superficial (SZ) to deep zone (DZ). (c) Overall relative fluorescence units (RFU) of exosome and cationic exosome uptaken by cartilage. The fluorescence intensities were quantified using ImageJ and normalized to the fluorescence intensity of native exosome in IL-1α treated cartilage. n=6. All data are presented as mean±s.d., and they are compared by one-way ANOVA with post-hoc Tukey's HSD test (** vs. respective Exo, p<0.01; **** vs. respective Exo, p<0.001).

FIG. 6 shows in vivo cartilage transport of Exo-CPC+14R in OA joint. (a) Scheme of in-vivo transport studies of AF647-labeled Exo and Exo-CPC+14R. Native and Exo-CPC+14R were IA-injected into both healthy and DMM mice knee joints 9 weeks post-surgery. Confocal images show the distribution of Exo and Exo-CPC+14R in cartilage layers of (b) healthy and (c) DMM joints on day 1 post-IA injection. Images displayed from left to right correspond to the lateral femoral side, medial femoral, and lateral tibial cartilage, respectively. Corresponding Safranin-O and H&E staining of joint sections are shown.

FIG. 7 shows cartilage targeted gene delivery using Exo-CPC+14R in vivo. (a) Schematic of mRNA loading inside Exo using lipofectamine 2000. (b) In-vitro eGFP mRNA expression in the HEK293t cells delivered by lipofectamine 2000, native Exos, and Exo-CPC+14R. (c) IA treatment regimen of eGFP mRNA loaded Exo and Exo-CPC+14R in DMM mice. (d) In-vivo GFP expression of Exo and Exo-CPC+14R on day 2. The green fluorescence of GFP protein was imaged using confocal microscopy. GFP monoclonal antibody staining also confirmed the GFP expression (brown). The black arrows assist in indicating the anti-GFP signal.

FIG. 8 shows exo-CPC+14R mediates enhanced eGFP mRNA expression in human OA cartilage. (a) Cartilage explants collected from human talus joints. (b) Experimental timeline of mRNA delivery via Exo/Exo-CPC+14R in IL-1α treated arthritic human cartilage model. (c) In-vitro GFP expression of mRNA delivered using Exo and Exo-CPC+14R in healthy and IL-1α treated human left talus cartilage explants from superficial (SZ) to deep zone (DZ). (d) Safranin-O and (e) H&E staining of healthy and IL-1α treated human talus cartilage sections.

FIG. 9 shows stability of cationic exosomes. Hydrodynamic size and zeta potential of Exo, Exo-PEG, and Exo-Av after one freeze-thaw cycle (at −80° C.) and the following 48 h culture at 37° C. n=4. Data are presented as mean±s.d., and they are compared by one-way ANOVA with post-hoc Tukey's HSD test (* vs. respective fresh, p<0.05).

FIG. 10 shows transport of native and cationic exosomes in healthy cartilage. Green fluorescence channels and merged confocal images of the native and cationic exosomes treated healthy cartilage explants. WGA staining (red) and DAPI (blue) were used to stain the chondrocyte membrane and its nucleus within the cartilage explant. Exosomes are labeled in green.

FIG. 11 shows transport of native and cationic exosomes in OA conditioned cartilage. The green fluorescence channels and merged confocal images of the native and cationic exosomes treated IL-1α challenged cartilage explants simulating early OA condition with an average of 35% GAG loss. WGA staining (red) and DAPI (blue) were used to stain the chondrocyte membrane and its nucleus within the cartilage explant. Exosomes are labeled in green.

FIG. 12 shows stability of Exo-Avidin. Hydrodynamic size of Exo-Avidin in PBS and culture media with the addition of 250 μM Tween 20. n=4. Data are presented as mean±s.d., and are compared by one-way ANOVA with post-hoc Tukey's HSD test (* vs. respective fresh, p<0.05).

FIG. 13 shows cartilage transport of Exo-CPC+14R in DMM mice joint. Distribution of Exo and Exo-CPC+14R in cartilage layers of (b) healthy and (c) DMM joints on day 3 confirmed by confocal microscopy. Images displayed from left to right correspond to the lateral femoral side, medial femoral and lateral tibial cartilage, respectively.

FIG. 14 shows the assessment of targeted delivery of mRNA loaded Exo-CPC+14R in human cartilage. In-vitro GFP mRNA expression delivered by Exo and Exo-CPC+14R in healthy and IL-1α treated human right talus cartilage explants from superficial (SZ) to deep zone (DZ).

### DETAILED DESCRIPTION OF THE INVENTION

This invention is based in part on the surprising discovery that exosomes can be modified to incorporate into their lipid bilayer membrane a positive surface charge using cationic peptide carriers (CPCs). This modification enables penetration of the modified exosomes into negatively charged tissue, such as cartilage, due to favorable electrostatic interactions.

In certain aspects, provided herein are modified exosome complexes, comprising:
(i) an exosome;
(ii) a linking moiety;
(iii) a polypeptide residue or a protein residue;
wherein the exosome comprises a lipid bilayer;
the linking moiety is linked to the lipid bilayer via non-covalent interactions; and
the protein residue or polypeptide residue is covalently linked to the linking moiety.

In certain embodiments, the linking moiety comprises a polymeric moiety. In further embodiments, the linking moiety comprises polyethylene glycol (PEG). In yet further embodiments, the linking moiety comprises a lipid moiety. In still further embodiments, the linking moiety comprises 1,2-distearoyl-sn-glycero-3-phosphoethanolamine (DSPE). In certain embodiments, the linking moiety comprises DSPE-PEG. In further embodiments, the non-covalent interaction is hydrophobic partitioning. In yet further embodiments, the linking moiety comprises a triazole moiety. In still further embodiments, wherein the linking moiety comprises a residue of dibenzocyclooctene. In certain embodiments, the linking moiety comprises DSPE-PEG-Biotin.

In certain embodiments, the complex comprises an exosome, a linking moiety, and a protein residue. In further embodiments, the protein residue is avidin.

In certain embodiments, the complex comprises an exosome, a linking moiety, and a polypeptide residue. In further embodiments, the polypeptide residue comprises 2 to 40 amino acid residues, and the net charge of the polypeptide is +7 to +20. In yet further embodiments, the polypeptide comprises at least one arginine residue, lysine residue, or other positively charged amino acid residue. In still further embodiments, the polypeptide comprises at least one arginine residue or lysine residue. In certain embodiments, the polypeptide comprises (i) a plurality of arginine residues, and a plurality of alanine residues, or (ii) a plurality of arginine residues, and a plurality of asparagine residues, or (iii) a plurality of arginine residues, and a mixture of alanine and asparagine residues; or (iv) a plurality of lysine residues, and a plurality of alanine residues; or (v) a plurality of lysine residues, and a plurality of asparagine residues; or (vi) a plurality of lysine residues, and a mixture of alanine and asparagine residues; or (vii) a plurality of arginine residues. In further embodiments, the net charge of the polypeptide residue is +7 to +14. In yet further embodiments, the net charge of the polypeptide is +8. In still further embodiments, the net charge of the polypeptide is +14. In certain embodiments, the polypeptide residue is selected from the group consisting of: AKAKAKAKAKAKAKANANAN; RRAAAARRAAAARRAAAARR; RRRRAARRRAARRRAARRRR; (ARRRAARA)₄; RRRRRRRRRRRRRRRRRRRR;

And RRRR(NNRRR)₃R. In certain embodiments, the polypeptide residue is RRRR(NNRRR)₃R.

In certain aspects, provided herein are methods of preparing a modified exosome complex, comprising:
(a) combining a linking moiety and an exosome comprising a lipid bilayer, thereby associating the linking moiety with the lipid bilayer of the exosome via non-covalent interactions;
(b) combining a polypeptide residue or protein residue with a buffer solution to neutralize the charge of the polypeptide residue or protein residue;
(c) combining the charge-neutralized polypeptide residue or protein residue with the linking moiety associated with the lipid bilayer of the exosome, thereby forming a covalent linkage between the charge-neutralized polypeptide residue or protein residue and the linking moiety; and
(d) bringing the buffer solution to physiological pH and salinity.

In certain embodiments, the neutralization of the charge of polypeptide residue or protein residue fosters efficient insertion of the residues via the hydrophobic portion of the linker moiety in the lipid bilayer of the exosome. In further embodiments, without neutralization the cationic motifs may go inside the exosome similar to lipofectamine. In yet further embodiments, the hydrophobic insertion happens at the isoelectric point of the polypeptide residue or protein residue. In still further embodiments, after the modified configuration of exosomes is made, the buffer is exchanged back to physiological salinity and pH.

In certain embodiments, the buffer solution further comprises a surfactant, such as Tween 20, Tween 60, Tween 80, SPAN 40, SPAN 60, Span 65, and Span 80, or combinations thereof. In further embodiments, the surfactant prevents aggregation and induced stability of the cationic exosomes. In yet further embodiments, the buffer solution further comprises Tween 20. In still further embodiments, the buffer solution further comprises between about 60 μM to about 250 μM Tween 20.

In certain aspects, provided herein are methods of preparing a modified exosome complex, comprising:
(i) combining a polypeptide residue or protein residue with a linking moiety, thereby forming a covalent linkage between the polypeptide residue or protein residue and the linking moiety;
(j) combining the polypeptide residue or protein residue with a buffer solution to neutralize the charge of the polypeptide residue or protein residue;
(k) combining the linking moiety and an exosome comprising a lipid bilayer, thereby associating the linking moiety with the lipid bilayer of the exosome via non-covalent interactions;
(l) bringing the buffer solution to physiological pH and salinity.

In certain embodiments, steps (j) and (k) occur simultaneously.

In certain aspects, provided herein are methods of encapsulating RNA into an exosome comprising:
(a) combining lipofectamine and a solution comprising RNA, thereby forming a first mixture;
(b) combining the first mixture and an exosome, thereby forming a second mixture; and
(c) combining the second mixture with RNase.

In certain embodiments, the exosome is the modified exosome complex. In further embodiments, the exosome is a native exosome.

In certain aspects, provided herein are methods of delivering a therapeutic agent to a negatively charged tissue, comprising administering to a subject in need thereof a therapeutically effective amount of a composition;
wherein the composition comprises a modified exosome complex; and a therapeutic agent.

In certain embodiments, the therapeutic agent is a nucleic acid, a protein, or a small molecule drug. In further embodiments, the therapeutic agent is a nucleic acid. In yet further embodiments, the nucleic acid comprises RNA or a plasmid vector. In still further embodiments, the RNA is an mRNA. In certain embodiments, the RNA is an siRNA. In further embodiments, the RNA is eGFP mRNA.

In certain embodiments, administering the composition comprises intra-articular injection. In further embodiments, administering the composition comprises oral administration. In yet further embodiments, administering the composition comprises transmucosal administration.

In certain embodiments, the negatively charged tissue is selected from the group consisting of cartilage, meniscus, tendons, ligaments, fracture callus, retina, intervertebral disc, mucosal membrane, and malignant tissue. In further embodiments, the negatively charged tissue is cartilage. In yet further embodiments, the negatively charged tissue is mucosal membrane.

In certain aspects, provided herein are methods of treating a joint disease, comprising administering to a subject in need thereof a therapeutically effective amount of a composition;
wherein the composition comprises a modified exosome complex; and a therapeutic agent.

In certain embodiments, the joint disease is selected from the group consisting of rheumatoid arthritis, spondyloarthritis, juvenile idiopathic arthritis, lupus, gout, bursitis, and osteoarthritis. In further embodiments, the joint disease is osteoarthritis.

In certain embodiments, the therapeutic agent is a nucleic acid, a protein, or a small molecule drug. In further embodiments, the therapeutic agent is a nucleic acid. In yet further embodiments, the nucleic acid comprises RNA or a plasmid vector. In still further embodiments, the RNA is an mRNA. In certain embodiments, the RNA is an siRNA. In further embodiments, the RNA is eGFP mRNA.

The intrinsic therapeutic potential of exosomes can be enhanced by increasing their joint residence time and by making them cartilage penetrating and binding such that they can reach their chondrocyte targets in tissue deep zones. The high negative fixed charge density (FCD) of cartilage offers a unique opportunity to utilize electrostatic interactions to enhance intra-tissue transport, uptake, and retention of exosomes by making them positively charged. Based on cartilage negative FCD, optimally charged protein and peptide-based cartilage targeting cationic motifs were designed (Table 1) that can rapidly penetrate through the full-thickness of cartilage in high concentrations by using weak and reversible electrostatic binding with the negatively charged cartilage GAGs following IA administration in the synovial joint. The cationic glycoprotein, Avidin, possessing optimal net size (<10 nm hydrodynamic diameter) and charge (between +6 and +20) demonstrated up to 180× higher uptake ratio (concentration of Avidin inside cartilage than surrounding fluid at equilibration), full thickness penetration and long-term retention inside rat and rabbit cartilage following IA injection. Based on Avidin's structure, arginine and lysine rich short length cartilage peptide carriers (CPCs) with varying net charges (from +7 to +14) and hydrophilicity were designed that have also shown high equilibrium intra-cartilage uptake in the range of 15-350×.

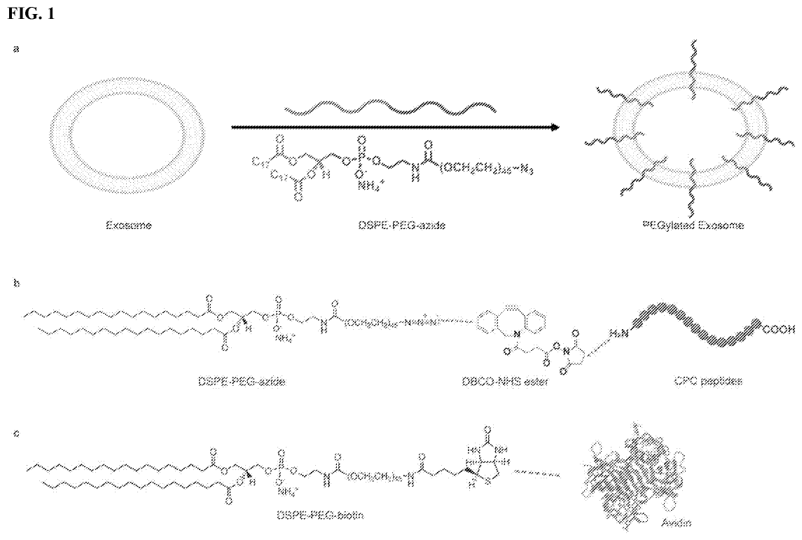

Here, engineering of surface-charge-reversed exosomes by anchoring cationic CPCs and Avidin to the lipid bilayer membrane via simple lipid insertion to enable their full-thickness penetration into cartilage at high concentrations is reported. Using either aqueous-based click chemistry or Avidin-biotin non-covalent binding, successful anchoring of cationic peptides and Avidin (FIG. 1) on exosome surface is demonstrated thereby reducing its overall negative charge. Since MSC-derived exosomes have a low yield and purity, exosomes isolated from bovine fat-free milk have been used instead (as described previously), which is an inexpensive and scalable source, as a model for surface modification.

The work demonstrates that cationic exosomes can effectively target negatively charged early-stage arthritic cartilage matrix than native anionic exosomes. Cationic exosomes penetrated through the full thickness of cartilage tissue and were uptaken by the chondrocytes residing in its deep layers. These exosomes also demonstrated efficient delivery of the encapsulated eGFP mRNA to the cartilage cells in a surgically induced destabilization of medical meniscus (DMM) mouse model as well as in cytokine-challenged human ankle cartilage explant model of early stages of OA. Exosomes are known to exhibit many desirable features of an ideal drug delivery system like long-circulating half-life, biocompatibility, and minimal toxicity. This validates the remarkable potential of cationic exosomes as natural, safe, cell-free carriers for the delivery of disease modifying gene materials for OA therapy.

### Discussion

Exosomes, a nanoscale subclass of extracellular vesicles secreted by cells, have emerged as a promising tool for drug delivery due to their non-immunogenic properties and specialized abilities in intercellular communication. Despite extensive research on evaluating the intrinsic therapeutic potential of exosomes derived from MSCs for cartilage repair, it remains unclear whether they can effectively penetrate the dense, highly negatively charged cartilage matrix to reach chondrocytes located in deeper tissue layers. To address this, a method of anchoring cartilage-targeting cationic motifs onto the exosome lipid bilayer to reverse its net negative charge using buffer pH as a charge reversal switch has been developed. The pH of the reaction buffer was brought close to the isoelectric points of cationic motifs (pH 8 and 9 for CPC+14R and Avidin, respectively) that neutralized the positive charge of these motifs enabling anchoring of 300-500 moles of cationic motifs per mole of exosome (Table 2). Exosomes have been shown to remain stable at pH<10 for up to 24 hours, allowing the use of reaction buffers at pH 8-9 for anchoring cationic motifs onto their membrane. This neutralized the net negative charge of exosomes without altering their size or morphology. Following the reaction, the buffer was exchanged back to physiological pH and salinity. This way, ionic crosslinking induced aggregation was minimized—a common problem encountered previously (e.g., attempt to use cationic pullulan to target injured liver). Cationic exosomes penetrated the full thickness of early to mid-stage arthritic cartilage and achieved high chondrocyte uptake, whereas unmodified native exosomes were incompetent in penetrating healthy or arthritic cartilage. These findings offer a promising new class of cell-free cartilage-targeting cationic exosomes with potential applications in drug and gene delivery to chondrocytes.

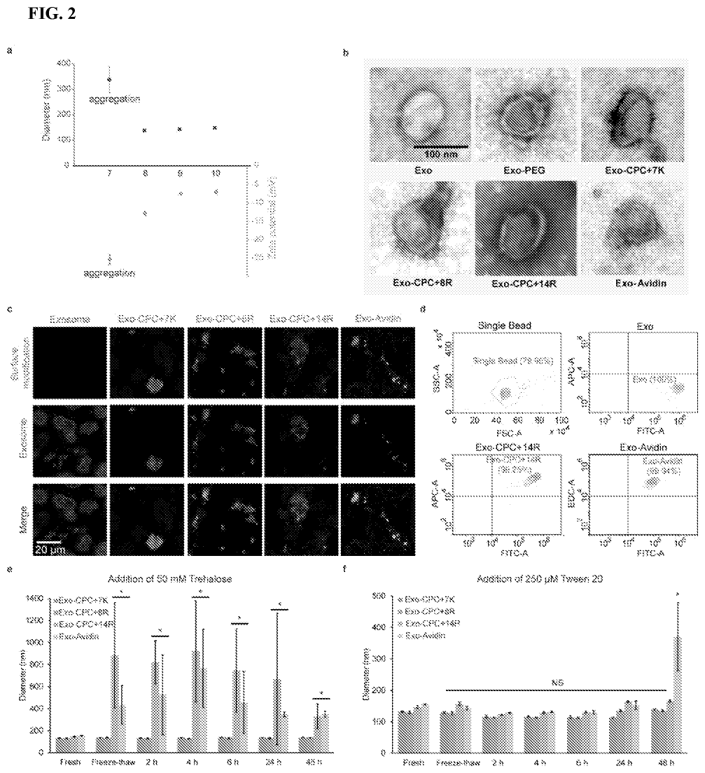

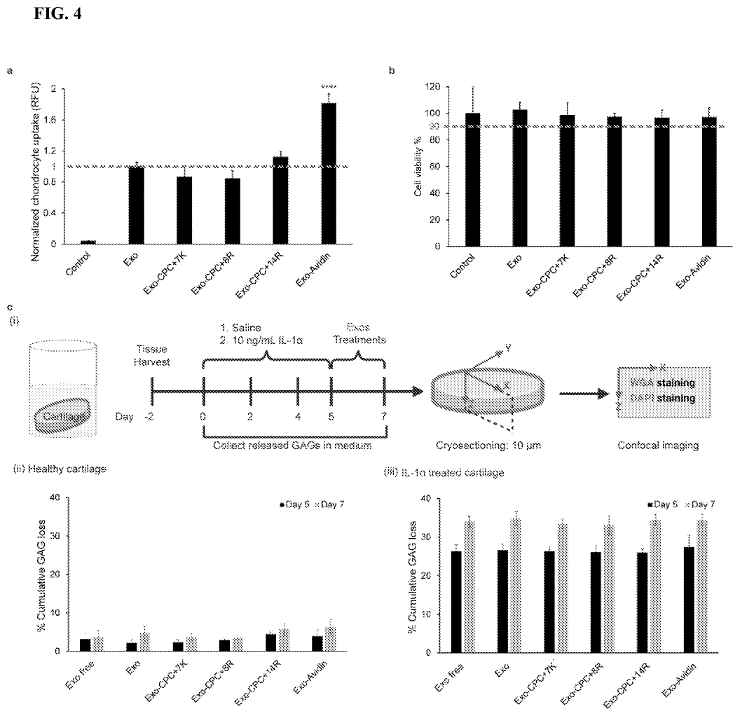

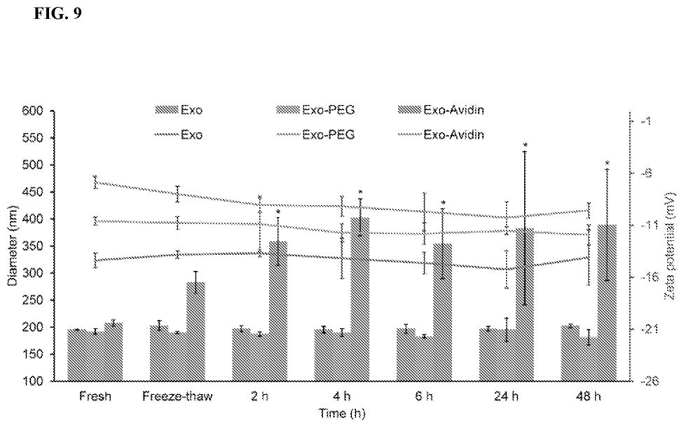

The negative net charge of exosomes contributes to their thermal stability at physiological conditions allowing for their long-term storage. Cationic Exo-Avidin started to form aggregates after one freeze-thaw cycle resulting in increased size at 37° C. (FIG. 9). This was an inevitable consequence of the reduced net negative charge. While PEG is commonly used to prevent aggregation of lipid nanoparticles (LNP), surface modification with a short length PEG₂₀₀₀ was not effective in preventing aggregation of cationic exosomes. The addition of low concentration non-ionic Tween 20 significantly inhibited aggregation after one freeze-thaw cycle and 24 h culture at 37° C. (FIG. 2) that can be attributed to Tween 20's ability to compete with aggregates for air-water interfaces. Genetic engineering has been widely used for cell membrane functionalization prior to exosome isolation for use in OA therapy. However, the expression and contents of membrane proteins in exosomes released from parent cells are sensitive to the culture environment. Another promising approach involves the fusion of exosomes with liposomes, however, concerns related to the integrity of exosome membrane and immunogenicity from liposomes limit its clinical potential. In comparison, the modular surface modification technique used here involves a physical insertion of lipophilic DSPE block into the lipid bilayer with high insertion stability, which minimizes disruption of membrane functional properties. Direct interactions between the cell membrane and cationic molecules may induce membrane pore formation and alter membrane fluidity, leading to cytotoxicity. Described herein, the coating of hydrophilic PEG from DSPE-PEG-azide on the exosome membrane reduced these undesired interactions between the membrane and cationic motifs, thereby mitigating any cytotoxicity concerns (as evident from the data in FIG. 4).

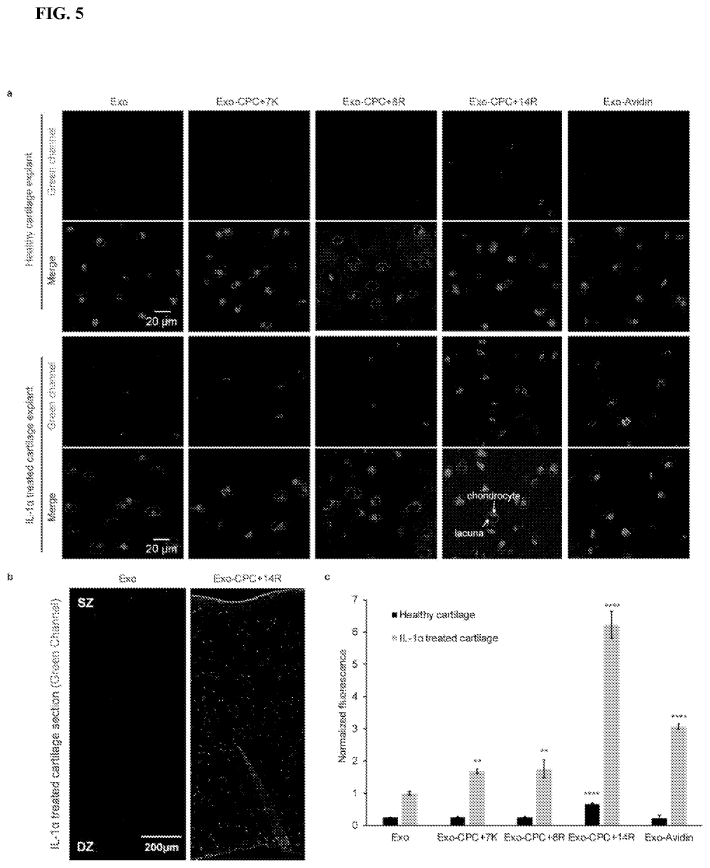

Of note, the mechanism by which cationic exosomes penetrate into the arthritic cartilage is not fully understood and requires further investigation. While electrostatic interactions are dominant, other factors such as changes in the ECM composition and increased pore size, and the role of specific cell surface receptors enabling adsorptive transcytosis cannot be ruled out. For example, Mauro Perretti and colleagues observed that neutrophil exosomes were only able to penetrate through the full thickness of IL-1β treated arthritic cartilage explants. In contrast, the same exosomes were unable to penetrate through the healthy cartilage explants, suggesting that the altered ECM of OA cartilage provides a conducive milieu for exosome transport. Interestingly, synthetic microcapsules of comparable size to exosomes were unable to penetrate through either healthy or IL-1β-treated cartilage, suggesting that exosome transport may not be mediated solely through passive diffusion. Described herein, although the conjugation of cationic motifs did not reverse the net macroscopic charge of exosomes to cationic, it created a positively charged delivery mechanism at microscale that could penetrate through the full thickness of cartilage and target chondrocytes in high concentrations. The zeta potential measurement only reflects the electrical potential at the slipping plane and cannot accurately represent the charge distribution on the surface layer of cationic exosomes. Prior work by Ribbeck and colleagues demonstrated that peptides with cationic and anionic amino acids arranged in blocks along the peptide length could partition up at the interface of negatively charged mucin barriers owing to Donnan effects, despite their net neutral electric charge. Similarly, Exo-CPC+14R with close to neutral zeta potential (FIG. 4), demonstrated high uptake by chondrocytes residing throughout the full thickness of early-stage arthritic cartilage (FIG. 5), reinforcing its potential use for delivery of genetic materials and macromolecules that rely on cell communication.

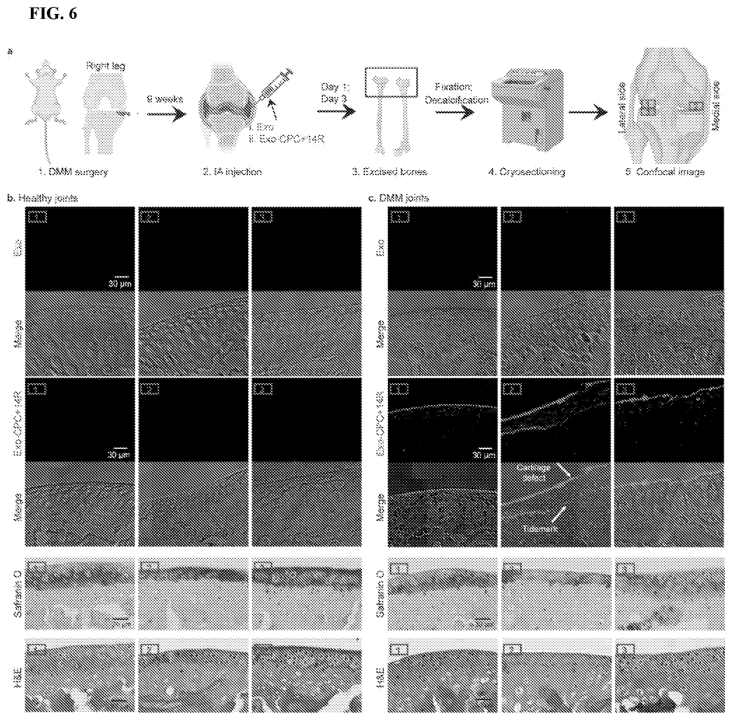

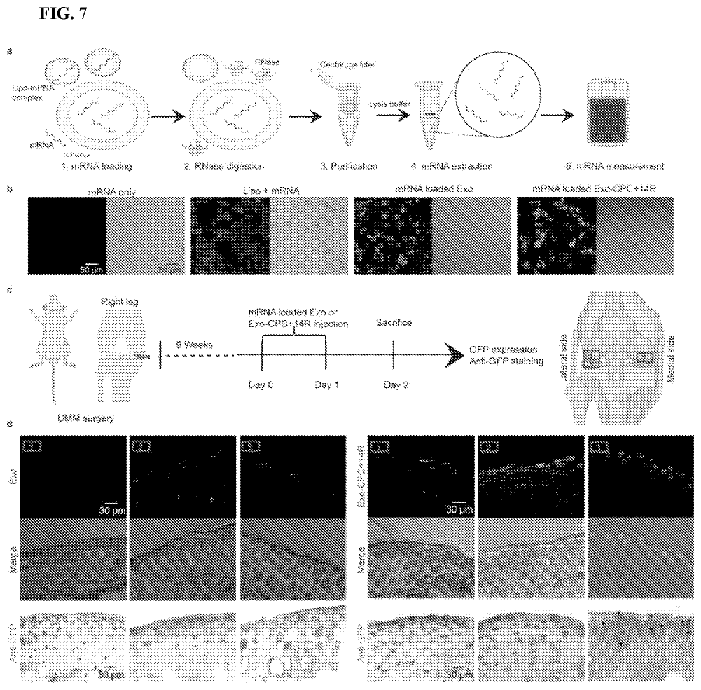

The advent of mRNA vaccines for COVID prophylaxis has paved way for mRNA therapy using synthetic LNPs in clinics. However, concerns around safety and immunogenicity of LNPs pose constraints on their long-term use for OA treatment. OA gene therapy has so far relied on adeno-associated viral vectors that are known to elicit undesired joint inflammation and other detrimental side effects. Exosomes are native lipid nanoparticles that are reported to possess intrinsic anti-inflammatory and immunosuppressive effects, making an ideal non-viral carrier alternative for gene delivery. While exosomes can encapsulate sufficient amounts of small interfering RNA (siRNA) and microRNA (range of 10-20 nt), loading of larger nucleic acids, like mRNA and CRISPR remains challenging. Here, cationic exosomes were leveraged to deliver eGFP mRNA (717 nt) through a simple exogenous loading technique and achieved higher GFP expression in HEK293t cells (FIG. 7b). Notably, cationic exosomes circumvented drug transport barriers and conferred a full-depth penetration of mRNA loaded Exo-CPC+14R in deep cartilage layers of both DMM mouse model and human talus cartilage despite their low loading efficiency (FIGS. 6-8).

This approach uses milk-derived exosomes due to their high yield, purity, and amenability to surface modification by post-insertion approach. Future work will focus on using MSC-derived exosomes with charge-reversal modification that can bestow a combinatorial effect for OA therapy due to their intrinsic therapeutic properties. The ability to maneuver the net charge of exosomes also offers the opportunity for designing targeted therapeutics for other tissues of varying net FCD. It is believed believe that cationic exosomes hold strong translational potential to create paradigm-shifting cartilage-targeted non-viral gene delivery approaches for OA therapy.

Exosomes are known to have intrinsic therapeutic potential and have recently been shown to be effective in tissue repair. Exosomes are emerging as a cell free regenerative therapy. Here, a new class of surface modified exosomes that are cationic in charge has been developed.

Exosomes have a negatively charged bilayer making it difficult to penetrate dense tissues like cartilage which is rich in aggrecan glycosaminoglycans. Similarly, a wide range of tissues exist that have negatively charged groups like proteoglycan, hyaluronic acid, anionic proteins etc. Some examples include musculoskeletal tissues like meniscus, tendon, ligaments, intervertebral discs, eye and tumors. Exosomes in their current form are ineffective in targeting these dense negatively charged tissues. A method for easy modification of the surface of exosomes to make them cationic is described. The chemistry enables modular surface properties such that any peptide or protein of interest can be added to the surface of exosome for efficient tissue targeting.

Provided herein is data using milk and mesenchymal stem cells (MSC) derived exosomes whose surface charge was neutralized or made slightly cationic enabling effective targeting of the mucosal membrane for oral drug delivery as well as for cartilage targeting for drug delivery applications. These exosomes are packed with genetic materials or anchored with protein drugs.

The present invention provides a new class of cationic exosomes and methods for synthesizing these cationic exosomes.

The chemistry presented herein enables modulation of the surface of exosomes, such that the properties of the exosomes may be tuned according to desired applications. Additionally, the data show excellent targeting and penetration of cartilage tissue which is a negatively charged tissue that remains a challenge in the field of drug delivery. By contrast, native (anionic) exosomes cannot target cartilage. The present invention also provides application of the exosomes in targeting mucosal membrane for oral delivery of biologics.

Drug delivery to cartilage remains challenging due to their rapid clearance from intra-articular joint space and hindered transport into cartilage deep layers due to its dense extracellular matrix (ECM) comprising of high density of negatively charged glycosaminoglycans (GAGs) and collagen II network. MSC derived exosomes could facilitate cartilage repair in OA animal models, but their large size (40˜200 nm) and negatively charged lipid bilayer (−20˜−25 mV) limited their penetration into deep layers of negatively charged cartilage. To solve these problems, the net charge on anionic exosomes has been reversed by anchoring their surfaces with cartilage targeting cationic peptide carriers (CPCs) and cationic glycoprotein Avidin. These cationic motifs were designed to effectively target cartilage based on its negative fixed charge density enabling ˜100-400× higher uptake than their neutral counterparts, full-thickness penetration, and long-term intra-cartilage retention. The hydrophobic tail of amphipathic DSPE-PEG (2 kDa)-azide (DPA) has been used for insertion into Exo lipid bilayer and the terminal azide for clicking cationic motifs enabling modular surface properties. About 300-500 cationic motifs were loaded per exosome resulting in reduced zeta potential of exosome from −25.4±1.3 mV to −2.5±1.5 mV. By making use of the charge interaction, these surface modified exosome showed fast penetration, high chondrocyte uptake and longer retention time in arthritis cartilages. Nucleus acids and proteins can be loaded in exosomes for intra-cartilage delivery.

As compared to prior technologies, the new class of neutral or cationic exosomes exhibit good thermal stability. Provided herein is a detailed method for synthesizing exosomes with varied net charge and storing the formulation long-term to avoid any aggregation issues.

Furthermore, the method laid out here enables synthesizing exosomes with varied net charge which is important for targeting a wide range of tissues with varying net negative fixed charge densities. This technique enables users (pharmaceutical/cell therapy companies) to make exosomes of any surface property and charge depending on their application and tissue target

As described herein, cartilage targeting cationic peptides (designed in the inventors' lab and showed be detailed in the patent) and proteins have been functionalized in different densities and demonstrate that these cationic exosomes can penetrate through the full thickness of cartilage in high concentrations while unmodified exosomes cannot. This discovery has the potential to transform the therapeutic space of cartilage repair and osteoarthritis.

Herein, simple and effective modular surface modification techniques have been designed for exosome membrane where any peptides or proteins of interest can be clicked, providing safe, cell free natural lipid carriers with intrinsic therapeutic potential for targeted drug delivery to cartilage and other negatively charged tissues.

Milk exosome harvest techniques have also been developed by applying casein chelation, differential ultracentrifugation and size-exclusion chromatography methods to obtain exosomes with high yield and high purity from the cheap, scalable resource.

This invention provides numerous advantages over known technologies, including enabling intra-cartilage targeting, reversing the net charge on anionic exosome, elevating chondrocyte uptake of surface modified exosome, enabling modular design of exosome surface using any peptides or proteins, enabling loading and delivery of nucleus acids, proteins and small molecular drugs, improving the stability of exosomes, and tuning a wide range of tissue targeting properties. The cationic exosomes described herein can target tissues due to electrostatic interactions. Native (negatively charged) exosomes cannot.

The invention described herein has numerous applications, including in intra-cartilage targeting, cell free tissue repair therapy, and delivery of nucleus acids, proteins, and small molecule drugs. Moreover, the drug delivery applications of the technology described herein can be extended to drug delivery in a wide range of negatively charged tissues like meniscus, intervertebral discs, mucosal membrane, and cancer tumors. The technology is also applicable to osteoarthritis treatment and various oral administration applications.

### Definitions

Unless otherwise defined, all technical and scientific terms used herein have the same meaning as commonly understood by one of ordinary skill in the art. Generally, the nomenclature used herein are well known and conventionally used in the art. The definition of main terms used in the detailed description of the invention is as follows.

The term “residue” as used herein refers to a portion of a chemical structure that may be truncated or bonded to another chemical moiety through any of its substitutable atoms. As an example, the structure of arginine is depicted below:

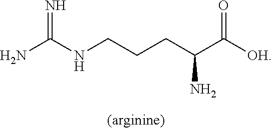 [SMILES: N=C(N)NCCC[C@H](N)C(=O)O]

“Nucleic acids,” as used herein, comprise a polymer of nucleotides (nucleotide monomers). Thus, nucleic acids are also referred to as polynucleotides. Nucleic acids may be or may include, for example, deoxyribonucleic acids (DNAs), ribonucleic acids (RNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-a-LNA having a 2′-amino functionalization), ethylene nucleic acids (ENA), cyclohexenyl nucleic acids (CeNA) and/or chimeras and/or combinations thereof. They may also include RNAi-inducing agents, RNAi agents, siRNAs, shRNAs, miRNAs, antisense RNAs, ribozymes, catalytic DNA, tRNA, RNAs that induce triple helix formation, aptamers, vectors, etc.

As used herein, the term “mRNA” refers to any RNA that encodes a (at least one) protein (a naturally-occurring, non-naturally occurring, or modified polymer of amino acids) and can be translated to produce the encoded protein in vitro, in vivo, in situ, or ex vivo.

As used herein, the term “siRNA” (small interfering RNA) means a short double-stranded RNA (dsRNA) that mediates efficient gene silencing in a sequence-specific manner.

As used herein, the term “plasmid vector” refers to a DNA structure able to insert exogenous DNA and capable of replicating in a recipient cell.

As used herein the term “exosome” refers to a cell-derived small (between 20-300 nm in diameter, more preferably 40-200 nm in diameter) vesicle comprising a membrane that encloses an internal space, and which is generated from said cell by direct plasma membrane budding or by fusion of the late endosome with the plasma membrane. The exosome is a species of extracellular vesicle. The exosome comprises lipid or fatty acid and polypeptide and optionally comprises a payload (e.g., a therapeutic agent), a receiver (e.g., a targeting moiety), a polynucleotide (e.g., a nucleic acid, RNA, or DNA), a sugar (e.g., a simple sugar, polysaccharide, or glycan) or other molecules. The exosome can be derived from a producer cell, and isolated from the producer cell based on its size, density, biochemical parameters, or a combination thereof.

The term “polypeptide” refers to an isolated polymer of amino acid residues, and are not limited to a minimum length unless otherwise defined. Peptides, oligopeptides, dimers, multimers, and the like, are also composed of linearly arranged amino acids linked by peptide bonds, and whether produced biologically and isolated from the natural environment, produced using recombinant technology, or produced synthetically typically using naturally occurring amino acids.

The term “negatively charged tissue” as used herein, comprises cartilage, meniscus, tendons, ligaments, fracture callus, retina, intervertebral disc, mucosal membrane, and malignant tissue.

The term “linker” as used herein refers to a group of atoms, e.g., 5-1,000 atoms, and can be comprised of the atoms or groups such as, but not limited to, carbon, amino, alkylamino, oxygen, sulfur, sulfoxide, sulfonyl, carbonyl, and imine. The linker can be attached to a modified nucleoside or nucleotide on the nucleobase or sugar moiety at a first end, and to a payload, e.g., detectable or therapeutic agent, at a second end. The linker may be of sufficient length as to not interfere with incorporation into a nucleic acid sequence. Examples of chemical groups that can be incorporated into the linker include, but are not limited to, an alkyl, an alkene, an alkyne, an amido, an ether, a thioether or an ester group. The linker chain can also comprise part of a saturated, unsaturated or aromatic ring, including polycyclic and heteroaromatic rings wherein the heteroaromatic ring may be an aryl group containing one to four heteroatoms, N, O or S. Specific examples of linkers include, but are not limited to, unsaturated alkanes, polyethylene glycols, and dextran polymers. For example, the linker can include, but is not limited to, ethylene or propylene glycol monomeric units, e.g., diethylene glycol, dipropylene glycol, triethylene glycol, tripropylene glycol, tetraethylene glycol, or tetraethylene glycol. In some embodiments, the linker can include, but is not limited to, a divalent alkyl, alkenyl, and/or alkynyl moiety. The linker can include an ester, amide, or ether moiety.

The term “lipid moiety” may include one or more PEG or PEG-modified lipids. Such species may be alternately referred to as PEGylated lipids. A PEG lipid is a lipid modified with polyethylene glycol. A PEG lipid may be selected from the non-limiting group consisting of PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

The lipid component of a linking moiety may include one or more phospholipids, such as one or more (poly)unsaturated lipids. Phospholipids may assemble into one or more lipid bilayers. In general, phospholipids may include a phospholipid moiety and one or more fatty acid moieties. A phospholipid moiety may be selected from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin. A fatty acid moiety may be selected from the non-limiting group consisting of lauric acid, myristic acid, myristoleic acid, palmitic acid, palmitoleic acid, stearic acid, oleic acid, linoleic acid, alpha-linolenic acid, erucic acid, phytanic acid, arachidic acid, arachidonic acid, eicosapentaenoic acid, behenic acid, docosapentaenoic acid, and docosahexaenoic acid. Non-natural species including natural species with modifications and substitutions including branching, oxidation, cyclization, and alkynes are also contemplated. For example, a phospholipid may be functionalized with or cross-linked to one or more alkynes (e.g., an alkenyl group in which one or more double bonds is replaced with a triple bond). Under appropriate reaction conditions, an alkyne group may undergo a copper-catalyzed cycloaddition upon exposure to an azide complex to a useful component such as a targeting or imaging moiety (e.g., a dye).

Phospholipids useful in the complexes and methods described herein may be selected from the nonlimiting group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycerophosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycerophosphocholine (DUPC), 1-palmitoyl-2-oleoyl-sn-glycero-3-phosphocholine (PO PC), 1,2-di-O-octadeceny 1-snglycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine(C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3 phosphoethanolamine (ME 16.0 PE), 1,2-distearoyl-sn-glycero-3-phosphoethanolamine, 1,2-dilinoleoy 1-sn-glycero-3-phosphoethanolamine, 1,2-dilinolenoyl-sn-glycero-3 phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin.

As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to a disease, disorder, and/or condition, to treat, improve symptoms of diagnose, prevent, and/or delay the onset of the disease, disorder, and/or condition.

As used herein, the term “therapeutic agent” refers to any agent that, when administered to a subject, has a therapeutic, diagnostic, and/or prophylactic effect and/or elicits a desired biological and/or pharmacological effect.

As used herein, the term “native exosome” refers to an unmodified exosome.

Surfactants useful in the methods of the invention include Tween 20 (polysorbate 20), Tween 60 (polysorbate 60), Tween 80 (polysorbate 80), SPAN 40 (sorbitan monopalmitate), SPAN 60 (sorbitan monostearate), Span 65 (sorbitan tristearate), and Span 80 (sorbitan monooleate).

### EXAMPLES

### Example 1—Synthesis of Surface-Modified Exosomes for Cartilage Targeting

Exosomes (Exos) were surface engineered by conjugating cationic motifs such as CPCs and Avidin on their surface (Table 1) to make them cartilage targeting.

TABLE 1
Sequence, molecular weight, and net
charge at pH 7 of cationic motifs.

| | | Molecular | | Net charge |
|---|---|---|---|---|
| Motifs | Sequence | weight | | at pH 7 |
| CPC + 7K | (AK)₇ANANAN | 1968 | Da | +7 |
| CPC + 8R | (RRAAAA)₃RR | 2049 | Da | +8 |
| CPC + 14R | RRRR(NNRRR)₃R | 2889 | Da | +14 |
| Avidin | / | 66 | kDa | +6 to +20 |

This was done by anchoring the hydrophobic part of DSPE-PEG linker into the Exo bilayer and conjugating CPCs or Avidin at the end of the linker either by using copper-free click chemistry or Avidin-Biotin binding as presented in FIG. 1. The zeta potential of Exos functionalized with CPC+7K and CPC+8R significantly increased to −12.7±0.5 mV and −11.8±1.6 mV respectively, compared to that of native Exo (−25.4±1.3 mV) by loading 383±31 and 415±41 moles of the respective peptides on each mole of Exo (Table 2).

TABLE 2
Diameter size (nm), zeta potential ξ (mV), polydispersity index (PDI)
and loading of cationic motifs on Exo membrane. Data are presented as mean ± s.d.

| | Exo | Exo-PEG | Exo-CPC + 7K | Exo-CPC + 8R | Exo-CPC + 14R | Exo-Avidin |
|---|---|---|---|---|---|---|
| Size (nm) | 136.7 ± 10.9 | 129.4 ± 2.8 | 132.4 ± 2.0 | 132.1 ± 2.7 | 146.9 ± 3.9 | 143.5 ± 6.1 |
| ξ (mV) | −25.4 ± 1.3 | −16.6 ± 0.8 | −12.7 ± 0.5 | −11.8 ± 1.6 | −2.5 ± 1.5 | −7.7 ± 0.4 |
| PDI (%) | 22.3 ± 0.6 | 15.9 ± 1.9 | 22.3 ± 0.8 | 18.3 ± 3.8 | 16.1 ± 4.5 | 24.0 ± 1.2 |
| Loading | / | 450 ± 55 | 383 ± 31 | 415 ± 41 | 494 ± 38 | 321 ± 63 |
| (motif:Exo) | | | | | | |

However, negatively charged Exo immediately formed aggregates when positively charged Avidin was added, owing to the formation of ionic crosslinks (FIG. 2a). To address the aggregation issues, the pH of the reaction buffer was increased from 7 to close to the isoelectric point of Avidin (˜10.5) to neutralize its charge. Successful change in zeta potential from −25.4±1.3 mV to −7.7±0.4 mV, along with no aggregation was observed at pH over 9. pH value of 9 was selected for the Exo-Avidin conjugation considering the stability of Exos in weak-alkaline conditions. Additionally, CPC+14R conjugation on Exos was achieved at pH 8 with a close to neutral zeta potential of −2.5±1.5 mV (Table 2). There were no significant differences in the hydrodynamic size of native Exos and the surface-modified Exos when measured using DLS. TEM images after uranyl acetate staining also showed the consistent ‘saucer-like’ morphology and similar diameter size between native Exos and surface-modified Exos (FIG. 2b). The ultrathin-shaded layers surrounding the Exo membrane represent the coating of DSPE-PEG-peptide or DSPE-PEG-Avidin. Previously, the dissociation constant (K_{D}) of the DSPE-PEG-azide to Exo lipid bilayer binding was confirmed to be about 347 M, which is consistent with the other reports of hydrophobic motif incorporation within a lipid bilayer. Using HEK293 cellular uptake, the successful surface modification of Exo was further confirmed using confocal microscopy (FIG. 2c). CPCs and Avidin were labeled with FITC, and Exos were labeled using the ExoGlow Red protein labeling kit. The overlap of green and red fluorescence uptaken by HEK293 confirmed the coating of CPCs or Avidin on the Exo membrane. Flow cytometry results also confirmed the successful coating of Exo membrane with CPC peptides and Avidin (FIG. 2d). This was demonstrated by the capture of dual fluorescence-labeled cationic Exosomes using anti-CD63 coated magnetic beads, which exhibited high fluorescence intensity in both FITC-A (Exo-FITC) and APC-A (CPC+14R-cy5) or EDC-A (Avidin-Texas Red) channels. To exclude the possibility that the cationic peptides or Avidin were absorbed on the Exo surface or uptaken by Exos, the FITC labeled CPCs or Avidin were mixed with Exos directly for 1 h at 37° C. in the absence of lipid insertion. After purification, the fluorescence of peptides or Avidin scarcely remained in the Exo solutions (data not shown), confirming that peptides or Avidin only bind to the Exo surface through the lipid insertion method.

It is worth mentioning that the more cationic formulations, Exo-Avidin and Exo-CPC+14R, formed aggregates in the PBS buffer measured by DLS method after one freeze-thaw cycle (FIG. 9). Trehalose and Tween 20 are commonly used cryoprotectants and protein stabilizers. In an attempt to prevent the aggregation of cationic Exos during storage, either 50 mM Trehalose or 250 μM Tween 20 was added to the PBS buffer (FIG. 2e, 2f). The addition of 50 mM Trehalose could not stabilize the cationic Exos after one freeze-thaw cycle. However, 250 μM Tween 20 in the buffer did keep the cationic Exos stable for 24 h at 37° C. after the freeze-thaw process. Therefore, 250 μM Tween 20 was added to the cationic Exo formulations for the following experiments.

### Exosome Harvest

Following the procedures recently reported, Exosomes (Exos) were harvested from pasteurized bovine skim milk. 108 mL of milk was diluted with 180 mL of PBS and then centrifuged at 3000 g for 15 min to eliminate cells, debris, and floating fat. 102 mL of the supernatant collected below the liquid surface layer was mixed with 0.25 M EDTA for 15 min on ice to chelate casein-calcium complexes. Exo pellets were collected following successive ultracentrifugation steps at 12,000 g, 35,000 g and 70,000 g for 1 h, and at 10,0000 g for another 2 h using the ultracentrifuge machine (Sorvall WX100, Thermo Fisher, Waltham, MA). Then Exo pellets were further purified by qEV10 35 nm SEC column.

### Lipid Insertion in Exosome Membrane

As shown in FIG. 1a, amphipathic DSPE-PEG-azide lipid was used for Exo membrane surface modification by inserting hydrophobic tail into the Exo lipid bilayer and exposing hydrophilic tail towards the outside aqueous environment. 25 μg/mL concentration of DSPE-PEG-azide solution was prepared by adding 50 μL of DSPE-PEG-azide DMSO solution (0.5 mg/mL) dropwise to 1 mL of PBS buffer to prevent the formation of micelles. 250 μL of 660 μg/mL Exo (protein concentration measured by BCA assay) was mixed with 1 mL of 25 μg/mL of DSPE-PEG-azide solution for 1 h at 37° C. The molar ratio of Exo to DSPE-PEG-azide was kept at 1:10000. 100 kDa MWCO centrifugal filter was used to purify the PEGylated Exo by removing excess DSPE-PEG-azide.

To calculate the loading of DSPE-PEG-azide on the Exo membrane, Exo was fluorescently labeled using the ExoGlow-Protein EV labeling Kit (Green) and DSPE-PEG-azide was labeled with DBCO-cy5. Using a standard curve, it was estimated that each mole of Exo would have about 450 55 moles of DSPE-PEG-azide inserted as described in previously.

### DSPE-PEG-Azide and CPC Peptide Conjugation

DBCO-NHS ester was introduced as a cross-linker to conjugate DSPE-PEG-azide and CPC peptides (FIG. 1b). DBCO, a reactive cycloalkyne, promotes alkyne-azide cyclo-addition, which is a copper-free click chemistry reaction. The NHS ester group can react rapidly with the primary amines of peptides to form the amide bond at pH 7-9. The sequences and net charges of CPCs are shown in Table 1. For CPC+8R and CPC+14R conjugation, 1 equivalent of CPC+8R (0.3 mg) or CPC+14R (0.4 mg) was dissolved in anhydrous DMF to make 5 mg/mL concentration. 5 equivalents of DBCO-NHS ester (0.28 mg) were dissolved in 56 μL of DMF and then mixed with peptide solution in a 2 mL glass vial. Following adjusting the pH of the solution to 7-9 using TEA, nitrogen gas was purged into the vial to remove air. This reaction was kept stirring at room temperature overnight. Since CPC+7K has multiple primary amines, the pH of the reaction was reduced to between 7 to 8 to primarily activate the N-terminus for site-specific conjugation. After the reaction, products were added to 2 mL of DI water to precipitate the unreacted DBCO-NHS ester. Following centrifugation at 8000 g for 5 min, the supernatant was collected and further purified by using a 3 kDa MWCO centrifuge filter. The purified products, DBCO-CPCs were lyophilized for subsequent conjugation with DSPE-PEG-azide. 0.34 mg of DSPE-PEG-azide (1.2 equivalent) was dissolved in 60 μL of DMSO and reacted with 1 equivalent of the lyophilized DBCO-CPC dissolved in 60 μL of DMSO. The reaction was kept stirring at room temperature overnight. Finally, synthesized DSPE-PEG-CPC was added dropwise to 13.6 mL of PBS buffer for Exo surface modification following the methods described above. During the purification procedure, free DSPE-PEG-CPC+7K and DSPE-PEG-CPC+8R were removed using a 100 kDa MWCO centrifuge filter. DSPE-PEG-CPC+14R, however, got stuck to the slightly negatively charged filter membrane (regenerated cellulose) owing to its high positive charge. SEC column (qEVoriginal/35 nm Gen 2 Column, Izon Science, New Zealand) was used as an alternative approach to purify the Exo-CPC+14R.

### DSPE-PEG-Biotin and Avidin Protein Conjugation

To synthesize Avidin functionalized Exos, DSPE-PEG-biotin was anchored on exosome surface instead of DSPE-PEG-azide (FIG. 1c). Then by using Avidin-biotin strong binding, Avidin was functionalized on the surface of Exos. As described above, DSPE-PEG-biotin was first inserted into Exo membrane by mixing Exo and DSPE-PEG-biotin (1:10000 molar ratio) at 37° C. for 1 h, which resulted in 450±55 moles of DPSE-PEG-Biotin per Exo⁵. To this, Avidin was added in 10:1 molar ratio of Avidin to DPSE-PEG-Biotin. Since Avidin is highly positively charged, it can form ionic complexes with negatively charged Exo bilayer resulting in aggregation. To avoid this, the conjugation was conducted in reaction buffers of increasing pH (7, 8, 9, and 10) up until the isoelectric point of Avidin (pI˜10.5). Excess Avidin after conjugation was removed using SEC column.

### Characterization of Cationic Exosomes

The size and zeta potential of cationic Exos were measured by Particle Analyzer (Litesizer 500, Anton Paar, Austria), and also confirmed by transmission electron microscope (TEM) using the negative staining method. Cationic Exos were dual-labeled such that Exo membrane was labeled with ExoGlow Red and cationic motifs (CPCs and Avidin) were labeled with FITC. Loading of cationic motifs on Exo surface was quantified by measuring fluorescence using a plate reader (Synergy H1, Biotek), which was then converted to concentration using respective standard curves. The presence of cationic motifs on Exo surface was also confirmed by evaluating uptake of dual labeled Exos in HEK293 cells. HEK293 cells were seeded at a density of 10,000 cells per well in a 96-well plate using a complete culture medium (high glucose DMEM supplemented with 10% FBS, 1% GlutaMAX, 1% nonessential amino acids, and 1% penicillin-streptomycin). After 24 h of cell, 50 μg of dual-labeled cationic Exos were added for 2.5 h. The cells were then imaged using a confocal microscope (LSM 800, ZEISS). To analyze the surface modification of Exo using flow cytometry, anti-CD63 coated magnetic beads were used to capture the Exos through the CD63 Exo-Flow Capture Kit following the manufacturer's protocol. The Exos were labeled with FITC, while CPC+14R and Avidin were labeled with Cy5 and Texas Red, respectively. Exos were surface modified with CPC+14R or Avidin and then attached to the beads. 10,000 beads were counted in every flow cytometry analysis and analyzed the forward scatter area (FSC-A) and side scatter area (SSC-A) to select single Exo-captured beads. The FITC-A, APC-A, and EDC-A channels showed the fluorescence intensities of FITC, Cy5, and Texas Red, respectively.

### Stability of Cationic Exosomes

The stability of cationic Exos in PBS and following freeze-thaw cycle was investigated. Cationic Exos were prepared in either 1 mL of PBS buffer, PBS with 50 mM trehalose as cryoprotectants or PBS with 250 μM Tween 20. Their size was measured using Particle Analyzer, following which these formulations were frozen at −80° C. for 24 h. Formulations were then thawed at room temperature, and their size was measured again. Then, these cationic Exo were kept at 37° C. and their size was measured again at 2, 4, 6, 24, and 48 h to evaluate their thermal stability.

### Example 2—Cationic Exosomes Exhibit Superior Cartilage Penetration and Retention Properties

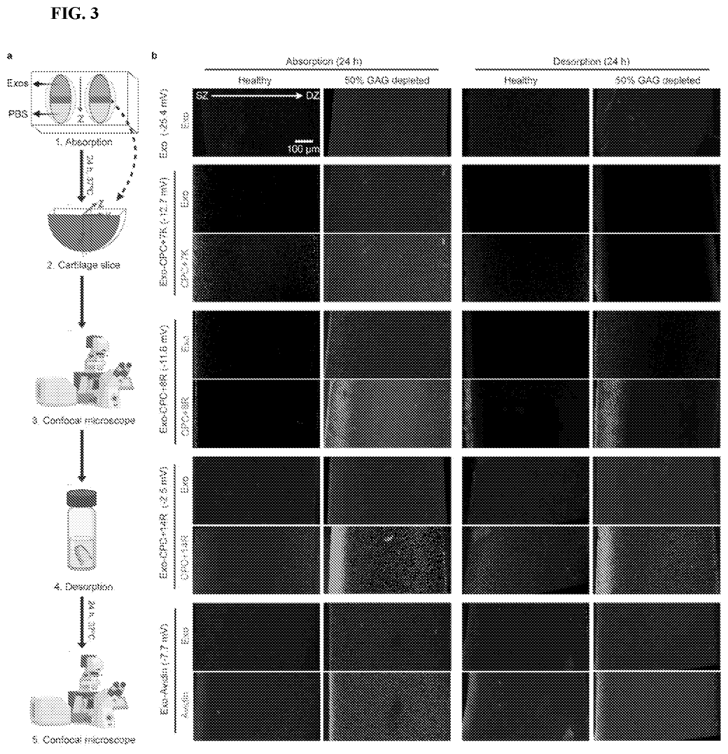

1D transport of cationic Exos from superficial (SZ) to deep zones (DZ) in both, healthy and arthritic cartilage explants over 24 h was investigated to evaluate their depth of penetration and retention (FIG. 3a). Consistent with other studies, the red fluorescence labeled native Exos could not penetrate through the healthy cartilage explants due to their large size (FIG. 3b). In trypsin induced 50% GAG depleted cartilage explants, native Exos showed penetration into deeper layers of the tissue potentially owing to enlarged pores in the matrix. Similarly, the dual-labeled cationic Exos did not penetrate through the full-thickness of healthy cartilage. As for transport in 50% GAG depleted cartilage explants, all the cationic Exos presented significantly higher fluorescence intensity than native Exos. Exo-CPC+14R, with close to neutral zeta potential, resulted in increased concentration at the SZ of cartilage, potentially owing to enhanced electrostatic binding with negatively charged GAGs. The cartilage explants equilibrated with different Exo formulations were then desorbed in PBS at 37° C. for 24 h and imaged again. The fluorescence intensities of native Exo, Exo-CPC+7K and Exo-CPC+8R in 50% GAG depleted cartilage showed significant reduction, demonstrating that most of them were released from cartilage within 24 h of desorption. It was consistent with previous reporting, which also showed that CPC+7K and CPC+8R had similar retention time inside cartilage due to weak charge-based binding interactions. On the contrary, no difference in the fluorescence intensities of Exo-CPC+14R and Exo-Avidin before and after desorption was observed, indicating that Exos modified with CPC+14R and Avidin had increased retention time inside the cartilage.

### Intra-Cartilage Transport and Retention of Cationic Exosomes

A custom-designed transport chamber (FIG. 3) was used to investigate the intra-tissue transport properties of cationic Exos in healthy, and 50% GAG-depleted bovine cartilage explants that simulate early to mid-stage OA condition. 6 mm diameter and 1 mm thick (6×1 mm) cartilage explants were harvested from the femoropatellar grooves of 2-to-3-week-old bovine knees (Research 87, Boylston, MA). Cartilage explants were treated with 0.1 mg/mL trypsin-EDTA for 4 h that resulted in 46.7±5.1% GAG loss, as measured using the DMMB assay. Half discs of healthy or 50% GAG depleted cartilage explants were glued at the center of the chamber. The side facing the explant's SZ was filled with 80 μL of 2.8 mg/mL of labeled Exo or dual-labeled cationic Exos, while the other side was filled with PBS. This transport setup was placed in a petri-dish containing water to minimize evaporation and placed on a shaker in an incubator at 37° C. for 24 h. The cartilage half discs were then sliced in the X-Z plane and imaged using a confocal microscope using a Z-stack and tile imaging. 555 nm Ex/618 nm Em and 488 nm Ex/525 nm Em wavelengths were used for ExoGlow Red and FITC respectively. Equilibrated cartilage explants were also desorbed in PBS for 24 h and then imaged again to estimate intra-cartilage retention.

### Example 3—Chondrocyte Uptake and Cytotoxicity of Cationic Exosomes

To investigate the chondrocyte uptake efficacy, cationic exosomes were labeled and incubated with primary bovine chondrocytes for 2.5 h. The uptake study confirmed that the conjugation of CPC+7K, CPC+8R, and CPC+14R on the Exo surface did not alter the innate affinity of Exos to be uptaken by the chondrocytes (FIG. 4a). Avidin, that has shown high chondrocyte uptake in our previous work, enabled 2× higher cellular uptake of Exo-Avidin compared to the native Exos. Considering the potential cytotoxicity of cationic Exos, the primary chondrocyte viability following 2.5 h treatment with 50 μg Exos and cationic Exos was evaluated using the MTT assay. All the cationic Exo groups demonstrated more than 90% cell viability similar to control and native Exo treatment groups, confirming no cytotoxicity concerns from cationic Exos (FIG. 4b). To further affirm the biocompatibility of cationic exosomes, as shown in FIG. 4c (i), fresh cartilage explants were treated with 10 ng/mL IL-1α for 5 days to create arthritic cartilage models that resulted in 26±0.6% GAG loss. Following the 2-day treatments with green fluorescence labeled native and cationic Exos, the GAG loss of IL-1α treated cartilage explants increased uniformly by about 10% (FIG. 4c (iii)), demonstrating that cationic Exo treatments did not induce more GAG loss compared to the native Exo treated or the Exo-free treated groups. The healthy cartilage explants resulted in about 5% GAG loss over 7 days when treated with native or cationic Exos confirming no cytotoxicity concerns associated with use of cationic Exos (FIG. 4c (ii)).

### Primary Chondrocyte Uptake and Cytotoxicity Analysis of Cationic Exosomes

Primary chondrocytes were collected from the femoral condyles of 2-to-3-week-old bovine knees (Research 87, Boylston, MA), following pronase and collagenase digestion as described before. Primary chondrocytes were seeded at a density of 20,000 cells per well in a 48-well plate using chondrocyte culture media (high glucose DMEM supplemented with 10% FBS, 1% GlutaMAX, 1% HEPES, 1% nonessential amino acids, 1% penicillin-streptomycin, 0.4% proline and 0.4% Ascorbic acid). Following cell culture for 24 h, 50 μg of ExoGlow-Green labeled Exos and cationic Exos were added for another 2.5 h. Flow cytometry (CytoFLEX, Beckman Coulter, CA) was used to quantify the fluorescence intensity of uptaken Exo by primary chondrocytes. In addition, chondrocytes treated with unlabeled Exo and cationic Exos for 2.5 h were analyzed using the MTT assay to evaluate cytotoxicity.

### Example 4—Cationic Exosomes can Target Chondrocytes Residing within the Full-Thickness of IL-1α Treated Cartilage Explants

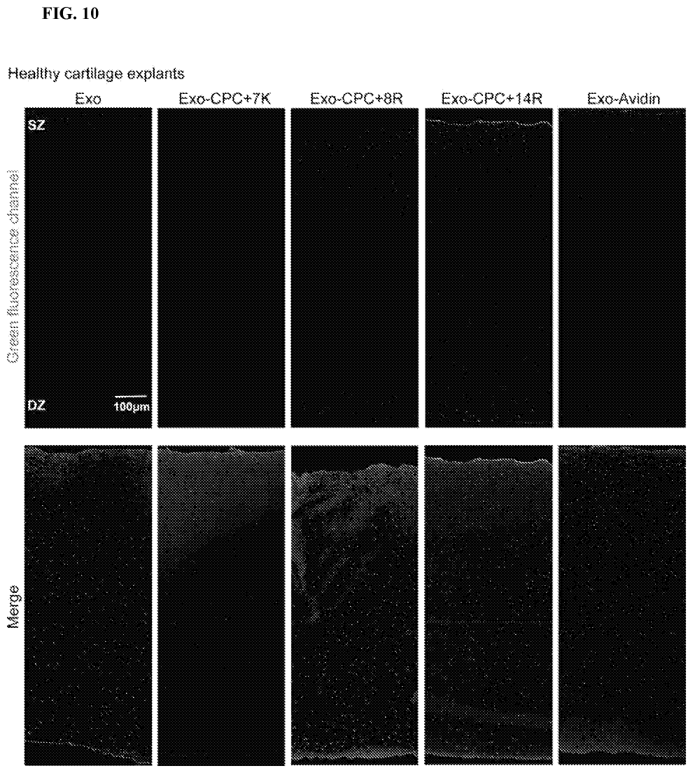

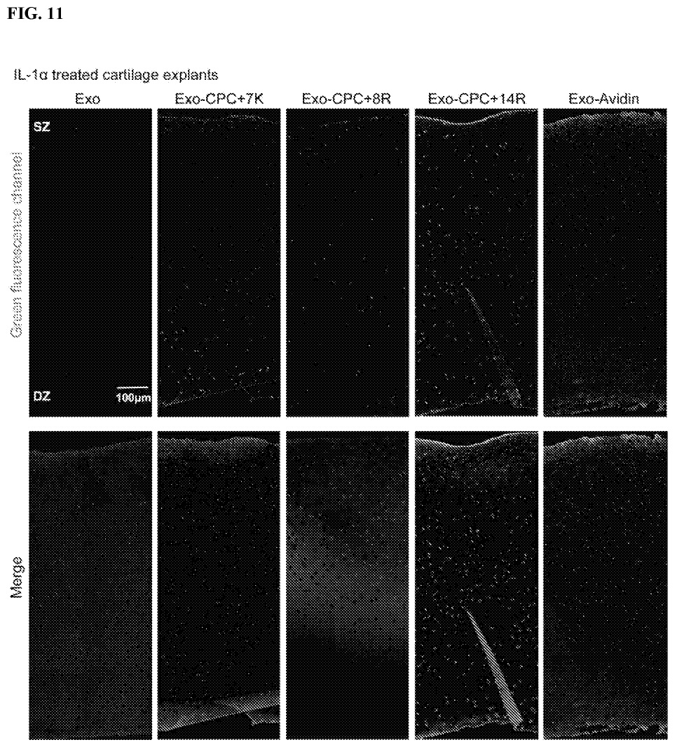

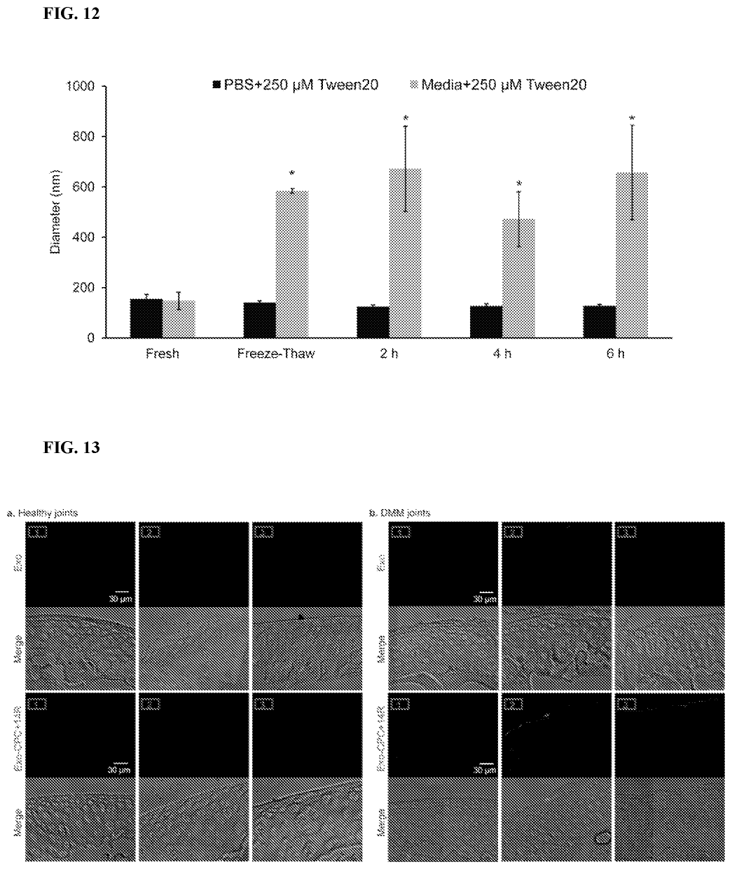

To investigate the targeted delivery of cationic exosomes to chondrocytes in the deep zone of cartilage, the Exos and cationic Exos were green fluorescence labeled in the experimental design as discussed in FIG. 4.c (i). These cartilage explants were stained with 4′,6-diamidino-2-phenylindole (DAPI, blue) and Wheat Germ Agglutinin (WGA, red) to identify the nucleus and chondrocyte membrane, respectively, on day 7. The cartilage explants treated with 10 ng/mL IL-1α resulted in 35% GAG loss, which emulates the cartilage breakdown in an early stage of OA. To prevent the margin effects of cartilage explants immersed in Exo baths, the central region of explants was selected as the area of interest for confocal imaging. No uptake of exosomes was observed in healthy cartilage explants, with the exception of Exo-CPC+14R, which was sparsely present (FIG. 5a-Healthy cartilage). IL-1α treated cartilage explants, on the other hand, showed significantly greater green fluorescence intensity when treated with cationic Exos compared to the native Exos (FIG. 5a—IL-1α treated cartilage). Exo-CPC+14R demonstrated the greatest chondrocyte (6× higher than native Exos) uptake, as evident from the high fluorescence intensity (also shown in the green channel images) that was widely distributed within the majority of chondrocytes. The difference between the uptake of native Exo and Exo-CPC+14R by the chondrocytes residing in the deep cartilage matrix layers can be further visualized in the green channel full-thickness images of the IL-1^{α} treated cartilage explants (FIG. 5b-5c). Exo-Avidin, unexpectedly, showed less than 2 times of chondrocyte uptake than Exo-CPC+14R within the full thickness of the IL-1α treated cartilage explant (FIG. 5c), which is different from our matrix transport (FIG. 3) and chondrocyte uptake data (FIG. 4a). To understand this, the stability of cationic Exo-Avidin in presence of cartilage culture media was assessed and compared to that in PBS in presence of 250 sM Tween 20 (FIG. 12). Data shows that cationic Exo-Avidin formed aggregates in the culture media despite the presence of Tween 20, explaining the lower uptake of Exo-Avidin compared to Exo-CPC+14R by the chondrocytes residing through the full-thickness of cartilage explant (FIG. 5a, 5c, FIG. 12). Other confocal images of cationic Exo treated full-thickness cartilage explants are presented in the FIGS. 10, 11 Exo-CPC+14R, which exhibited superior chondrocyte uptake in the full thickness of cartilage explants, was selected over Exo-Avidin for subsequent studies.

### Transport and Chondrocyte Uptake of Cationic Exosomes in Healthy and IL-1α Treated Cartilage Explants

3 mm diameter×1 mm thick bovine cartilage explants were cultured with 10 ng/mL of IL-1α for 5 days that resulted in 26±0.6% GAG loss, simulating an early-stage arthritic condition. A healthy cartilage model was developed by culturing bovine cartilage explants in IL-1α-free media for 5 days. The healthy and arthritic cartilage explants were treated with 150 μg of ExoGlow-Green labeled native and cationic Exos for 2 days. Subsequently, these cartilage explants were fixed using 4% paraformaldehyde (PFA) for 24 h and dehydrated in 15% and 30% sucrose solution for 8 h. The processed cartilage explants were then embedded in optimal cutting temperature compound and cryo-sectioned in 10 μm thick sections. These sections were then stained with DAPI and WGA to visualize the nucleus and chondrocyte membrane respectively.

### Example 5—Cationic Exosomes Transported Through the Full Thickness of DMM Mice Cartilage while Unmodified Native Exosomes Did not

To compare in vivo cartilage transport between native and cationic exosomes, animals were subjected to intra-articular (IA) injection post-9-week DMM surgery (FIG. 6a). The merged images (AF647 channel and bright field) of healthy mouse tibial and femoral bones in FIG. 6b show intact cartilage that did not uptake any unmodified Exo or cationic Exo-CPC+14R (as shown by the confocal fluorescence images). This is consistent with the in vitro results presented in FIGS. 3 and 5. Alexa Fluor 647 (AF647) labeled cationic Exo-CPC+14R, however, presented high fluorescence signal through the full-thickness layer of cartilage at 24 h following IA administration into the DMM mice joints (FIG. 6c). The medial femoral cartilage which suffered more burden than lateral femoral cartilage, exhibited obvious cartilage damage and GAG loss that facilitated Exo-CPC+14R to reach the cartilage tidemark. In the absence of electrostatic interaction, native Exos could not permeate into the DMM joint cartilage. Additional confocal images of Exo and Exo-CPC+14R transport in healthy and DMM mice joints at Day 3 are shown in FIG. 13. Although the fluorescence intensity of Exo-CPC+14R in the DMM cartilage decreased significantly due to elimination and degradation, some Exo-CPC+14R was still present in the superficial layers of cartilage.

### In Vivo Transport Studies of Exo and Exo-CPC+14R in Healthy and DMM Mouse Joints

All animal experiments were approved by the Institutional Animal Care and Use Committee at Rush University Medical Center. Destabilization of the medial meniscus (DMM) were performed in the right knee of 10-week-old male C57BL/6 mice purchased from the Jackson Laboratory (Bar Harbor, ME). As shown in FIG. 6a, 9 weeks after the DMM surgery, 10 μL of AF647 labeled Exo or Exo-CPC+14R (25 pg) were IA injected into both left (contralateral naïve) and right (DMM) knee joints of mice. At Day 1 and Day 3 post-administration, Exo and Exo-CPC+14R treated mice were sacrificed and the tibial and femur bones were excised. After 24 h fixation in 4% PFA, these bones were decalcified for 5 days using 14% EDTA, and then embedded into optimal cutting temperature medium. The tibial and femur bones were cryo-sectioned into 10 μm slides in coronal plane and imaged using confocal microscope at 650 nm excitation and 665 nm emission wavelengths. N=5 animals were used per treatment condition; a total of 10 animals were used for this study. The cryo-sections of tibial and femur bones were stained with 0.5% Safranin 0, 0.02% Fast Green and Weigert's iron hematoxylin for GAG detection (Safranin O staining). Structural changes in articular cartilage were documented in tissues stained with hematoxylin and eosin (H&E staining).

### Example 6—mRNA-eGFP Gene was Expressed in DMM Mice Cartilage Through Cationic Exosome Delivery

As a proof of concept, the application of Exo and Exo-CPC+14R for gene delivery were investigated by loading eGFP mRNA as a model gene. To that end, mRNA-eGFP loaded Exo/Exo-CPC+14R were synthesized (FIG. 7a) with a loading efficiency of 19.54±1.03%. The in vitro transfection efficiency was performed by incubating mRNA-loaded exosomes with HEK293T, and the cells were imaged for eGFP expression at 24 h post-transfection. In FIG. 7b, the mRNA-loaded Exo and Exo-CPC+14R induced high levels of GFP expression with robust fluorescence intensity. Noticeably, the GFP expression was higher in cells treated with Exo-CPC+14R compared to mRNA-lipofectamine complex, indicating that the cationic exosomes mediated a functional mRNA delivery to cells.

Subsequently, mRNA-eGFP loaded Exo and Exo-CPC+14R were intra-articularly injected into the joints of DMM mice to investigate gene delivery and expression efficiency in vivo (FIG. 7c). Exo-CPC+14R enabled distinct GFP fluorescence expression in chondrocytes residing in deep cartilage layers. In contrast, minimal GFP was expressed in DMM cartilage when using native Exo for eGFP mRNA delivery. The same results were confirmed by immunocytochemistry images using anti-GFP monoclonal antibody to identify the locations of GFP proteins.

mRNA Loading Strategy in Exosome

eGFP mRNA was loaded into Exo and Exo-CPC+14R with the help of lipofectamine (Lipo) 2000 transfection reagent. 1.5 μg eGFP mRNA and 7.5 μL of transfection reagent were diluted separately in Opti-MEM and incubated for 5 minutes. The two mixtures were then combined at a ratio of 1:1, followed by incubation at room temperature for 20 minutes to form mRNA-Lipo complexes. Exo and Exo-CPC+14R (125 μg) were added dropwise to the mRNA-Lipo solution and incubated at 37° C. for 30 minutes. The solution was then subjected to RNase digestion for 45 minutes at 37° C. to remove unencapsulated eGFP. The mRNA-eGFP loaded Exo and Exo-CPC+14R were purified and collected by centrifugation at 3000 g for 15 minutes using a 100 kDa MWCO centrifuge filter. To quantify mRNA loading efficiency, RNA was extracted from exosomes using a RNeasy Mini kit according to the manufacturer's protocol. The amount of isolated RNA was then measured using Qubit 4 Fluorometer (Thermo Fisher Scientific, Waltham, MA) with Qubit™ RNA BR assay kit.

In Vivo mRNA Expression Delivered by Exo and Exo-CPC+14R in Healthy and DMM Mouse Joints

The DMM mouse model was created as above. As shown in FIG. 7c, 10 μL of mRNA-eGFP loaded Exo and Exo-CPC+14R (25 μg) were intra-articularly injected into the DMM-operated legs of mice on day 0, respectively. The same doses of mRNA-eGFP loaded Exo and Exo-CPC+14R were intra-articularly injected again on day 1. On the next day, these mice were sacrificed, and the tibial and femur bones were excised for cryo-sectioning. The green fluorescence expression of GFP in the cartilage was observed under a confocal microscope. Furthermore, the sections were stained with a monoclonal antibody against GFP and subsequently visualized using a Vectastain Elite Rabbit IgG Detection Kit.

In-Vitro mRNA-eGFP Gene Expression

HEK293T cells were cultured in high glucose DMEM (supplemented with 10% FBS, 1% nonessential amino acids, and 1% GlutaMAX). For in-vitro eGFP transfection, cells were seeded at a density of 20,000 cells/well in a 96-well plate and subjected to overnight incubation. On the day of transfection, cells were treated with Exo and Exo-CPC+14R containing 200 ng eGFP mRNA in reduced serum media for 4 h, after which the cells were replenished with fresh DMEM. At 24 h post-transfection, the cells were imaged for green fluorescence to evaluate the eGFP mRNA expression using a confocal microscope.

### Example 7—Cationic Exo-CPC+14R Enabled Effective mRNA Expression in Human Arthritic Cartilage Explants

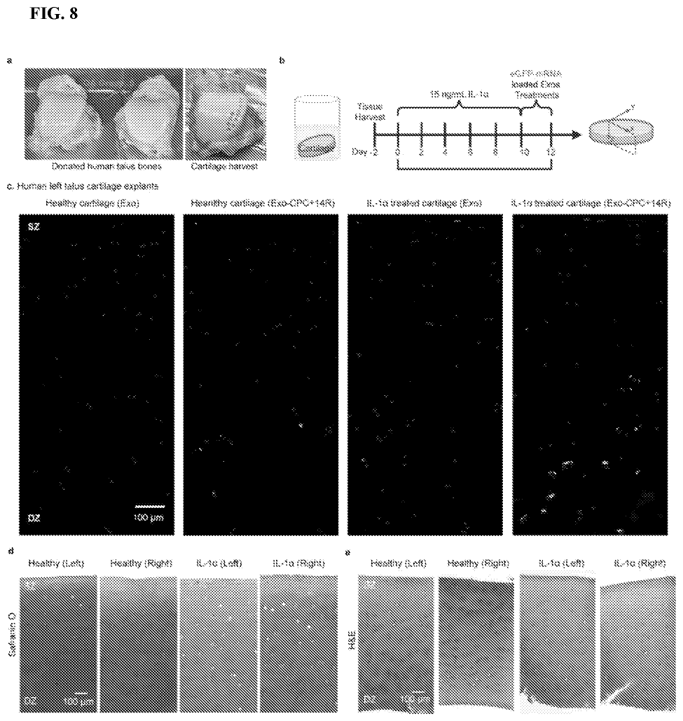

Arthritis was induced in healthy human cartilage explants by treating them with 15 ng/mL of IL-1α for a period of 10 days. In comparison to bovine cartilage, human cartilage exhibited lower sensitivity to IL-1α, resulting in only 19±1.1% GAG loss when challenged with higher concentrations of IL-1α and longer culture duration. Safranin 0 and H&E staining in FIG. 8e, 8f effectively demonstrated the extent of GAG loss and changes in the cartilage matrix during the early stages of arthritic human cartilage.

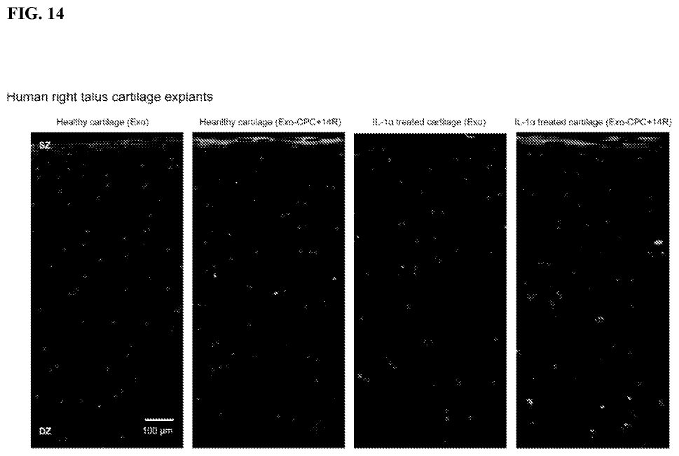

As anticipated, minimal GFP fluorescence was observed in healthy and IL-1α-treated human cartilage explants when treated with native Exos. In contrast, Exo-CPC+14R exhibited some GFP fluorescence in healthy human cartilage but enabled a significantly higher GFP expression in human chondrocytes mainly located in the DZ of IL-1α-treated human cartilage explants. This could be potentially due to stronger binding interactions of cationic exosomes with the remaining GAGs in the deep layers of cytokine treated cartilage explants (FIG. 8c). No difference in transport of Exo and Exo-CPC-14R or the induced GFP expression was observed in cartilage explants derived from left vs. right talus (FIG. 14).

eGFP mRNA Expression in Human Arthritic Cartilage Explants Using Cationic Exosomes

Left and right talus bones were obtained within 24 h (from donor 24 years of age, Hispanic, male) from the Gift of Hope Organ Bank. As shown in FIG. 8a, the cartilage surfaces from donor were scored as Collin grade 0.3 mm diameter with 1 mm thickness cartilage explants were harvested using a sterile punch as described before and then equilibrated in serum free culture media containing 95.2% high-glucose DMEM, 1.0% ITS, 1.0% HEPES, 1.0% NEAA, 1.0% PSA, 0.4% proline and 0.4% ascorbic acid for 48 h at 37° C., 5% CO₂. After a 2-day equilibration period, healthy human cartilage explants were cultured in a medium containing 150 μg Exo or Exo-CPC+14R encapsulating approximately 350 ng of eGFP mRNA. Following a 2-day culture, the treated cartilage explants were cryo-sectioned with a thickness of 10 μm using the aforementioned method. The human chondrocyte nucleus was stained using Hoechst. Additionally, other human cartilage explants were treated with 15 ng/mL of IL-1α for 10 days to simulate early-stage arthritic cartilage (FIG. 8b). The GAG loss and collagen matrix changes between healthy and IL-1α treated cartilage explants were compared using Safranin O staining and H&E staining. Subsequently, 350 ng eGFP mRNA loaded Exo and Exo-CPC+14R were added to the IL-1α challenged cartilage explants, respectively, for a 2-day culture period. Afterwards, these cartilage explants were cryo-sectioned and Hoechst stained.

### REFERENCES

1 Sellam, J. & Berenbaum, F. The role of synovitis in pathophysiology and clinical symptoms of osteoarthritis. Nat Rev Rheumatol 6, 625-635, doi:10.1038/nrrheum.2010.159 (2010).
2 Bajpayee, A. G. & Grodzinsky, A. J. Cartilage-targeting drug delivery: can electrostatic interactions help? Nat Rev Rheumatol 13, 183-193, doi:10.1038/nrrheum.2016.210 (2017).
3 He, T. et al. Hyaluronic Acid-Based Shape-Memory Cryogel Scaffolds for Focal Cartilage Defect Repair. Tissue Eng Part A 27, 748-760, doi:10.1089/ten.TEA.2020.0264 (2021).
4 Evans, C. H., Kraus, V. B. & Setton, L. A. Progress in intra-articular therapy. Nat Rev Rheumatol 10, 11-22, doi:10.1038/nrrheum.2013.159 (2014).
5 Warren, M. R. et al. Milk exosomes with enhanced mucus penetrability for oral delivery of siRNA. Biomaterials Science, doi:10.1039/DOBM01497D (2021).
6 Colombo, M., Raposo, G. & Théry, C. Biogenesis, secretion, and intercellular interactions of exosomes and other extracellular vesicles. Annu Rev Cell Dev Biol 30, 255-289, doi:10.1146/annurev-cellbio-101512-122326 (2014).
7 Toh, W. S., Lai, R. C., Hui, J. H. P. & Lim, S. K. MSC exosome as a cell-free MSC therapy for cartilage regeneration: Implications for osteoarthritis treatment. Semin Cell Dev Biol 67, 56-64, doi:10.1016/j.semcdb.2016.11.008 (2017).
8 De Bari, C. & Roelofs, A. J. Stem cell-based therapeutic strategies for cartilage defects and osteoarthritis. Curr Opin Pharmacol 40, 74-80, doi:10.1016/j.coph.2018.03.009 (2018).
9 Zhu, Y. et al. Comparison of exosomes secreted by induced pluripotent stem cell-derived mesenchymal stem cells and synovial membrane-derived mesenchymal stem cells for the treatment of osteoarthritis. Stem Cell Research & Therapy 8, 64, doi:10.1186/s13287-017-0510-9 (2017).
10 Bao, C. & He, C. The role and therapeutic potential of MSC-derived exosomes in osteoarthritis. Archives of Biochemistry and Biophysics 710, 109002, doi:https://doi.org/10.1016/j.abb.2021.109002 (2021).
11 Yan, C. et al. MicroRNA regulation associated chondrogenesis of mouse MSCs grown on polyhydroxyalkanoates. Biomaterials 32, 6435-6444, doi:https://doi.org/10.1016/j.biomaterials.2011.05.031 (2011).
12 Mao, G. et al. Exosomes derived from miR-92a-3p-overexpressing human mesenchymal stem cells enhance chondrogenesis and suppress cartilage degradation via targeting WNT5A. Stem Cell Research & Therapy 9, 247, doi:10.1186/s13287-018-1004-0 (2018).
13 Qiu, M., Liu, D. & Fu, Q. MiR-129-5p shuttled by human synovial mesenchymal stem cell-derived exosomes relieves IL-1β induced osteoarthritis via targeting HMGB1. Life Sciences 269, 118987, doi:https://doi.org/10.1016/j.1fs.2020.118987 (2021).
14 Vedadghavami, A., Zhang, C. & Bajpayee, A. G. Overcoming negatively charged tissue barriers: Drug delivery using cationic peptides and proteins. Nano Today 34, 100898, doi:10.1016/j.nantod.2020.100898 (2020).
15 Liang, Y. et al. Chondrocyte-specific genomic editing enabled by hybrid exosomes for osteoarthritis treatment. Theranostics 12, 4866-4878, doi:10.7150/thno.69368 (2022).
16 Headland, S. E. et al. Neutrophil-derived microvesicles enter cartilage and protect the joint in inflammatory arthritis. Sci Transl Med 7, 315ra190, doi:10.1126/scitranslmed.aac5608 (2015).
17 Wang, Y. et al. Exosomes from embryonic mesenchymal stem cells alleviate osteoarthritis through balancing synthesis and degradation of cartilage extracellular matrix. Stem Cell Res Ther 8, 189, doi:10.1186/s13287-017-0632-0 (2017).
18 Bajpayee, A. G. et al. Sustained intra-cartilage delivery of low dose dexamethasone using a cationic carrier for treatment of post traumatic osteoarthritis. Eur Cell Mater 34, 341-364, doi:10.22203/eCM.v034a21 (2017).
19 Vedadghavami, A. et al. Cartilage penetrating cationic peptide carriers for applications in drug delivery to avascular negatively charged tissues. Acta Biomater 93, 258-269, doi:10.1016/j.actbio.2018.12.004 (2019).
20 Vedadghavami, A. et al. Charge-based drug delivery to cartilage: Hydrophobic and not electrostatic interactions are the dominant cause of competitive binding of cationic carriers in synovial fluid. Acta Biomaterialia, doi:https://doi.org/10.1016/j.actbio.2022.08.010 (2022).
21 He, T. et al. Multi-arm Avidin nano-construct for intra-cartilage delivery of small molecule drugs. J Control Release 318, 109-123, doi:10.1016/j.jconrel.2019.12.020 (2020).
22 Vedadghavami, A., Hakim, B., He, T. & Bajpayee, A. G. Cationic peptide carriers enable long-term delivery of insulin-like growth factor-1 to suppress osteoarthritis-induced matrix degradation. Arthritis Research & Therapy 24, 172, doi:10.1186/s13075-022-02855-1 (2022).
23 Bajpayee, A. G., Wong, C. R., Bawendi, M. G., Frank, E. H. & Grodzinsky, A. J. Avidin as a model for charge driven transport into cartilage and drug delivery for treating early stage post-traumatic osteoarthritis. Biomaterials 35, 538-549, doi:10.1016/j.biomaterials.2013.09.091 (2014).
24 He, T. et al. Intra-articular kinetics of a cartilage targeting cationic PEGylated protein for applications in drug delivery. Osteoarthritis Cartilage 31, 187-198, doi:10.1016/j.joca.2022.09.010 (2022).
25 Zhang, C., Vedadghavami, A., He, T., Charles, J. F. & Bajpayee, A. G. Cationic Carrier Mediated Delivery of Anionic Contrast Agents in Low Doses Enable Enhanced Computed Tomography Imaging of Cartilage for Early Osteoarthritis Diagnosis. AC SNano 17, 6649-6663, doi:10.1021/acsnano.2c12376 (2023).
26 Bajpayee, A. G., Scheu, M., Grodzinsky, A. J. & Porter, R. M. A rabbit model demonstrates the influence of cartilage thickness on intra-articular drug delivery and retention within cartilage. J Orthop Res 33, 660-667, doi:10.1002/jor.22841 (2015).
27 Mehta, S. et al. Sustained intra-cartilage delivery of interleukin-1 receptor antagonist using cationic peptide and protein-based carriers. Osteoarthritis and Cartilage, doi:https://doi.org/10.1016/j.joca.2023.01.573 (2023).
28 Zhang, C., He, T., Vedadghavami, A. & Bajpayee, A. G. Avidin-biotin technology to synthesize multi-arm nano-construct for drug delivery. MethodsX 7, 100882, doi:10.1016/j.mex.2020.100882 (2020).
29 He, T., Zhang, C. & Bajpayee, A. G. in Biomedical Engineering Technologies: Volume 2 (eds Avraham Rasooly, Houston Baker, & Miguel R. Ossandon) 537-553 (Springer US, 2022).
30 Zhang, C. & Bajpayee, A. G. in Biomedical Engineering Technologies: Volume 1 (eds Miguel R. Ossandon, Houston Baker, & Avraham Rasooly) 797-812 (Springer US, 2022).
31 Angelova, M. I. et al. pH sensing by lipids in membranes: The fundamentals of pH-driven migration, polarization and deformations of lipid bilayer assemblies. Biochimica et Biophysica Acta (BBA)—Biomembranes 1860, 2042-2063, doi:https://doi.org/10.1016/j.bbamem.2018.02.026 (2018).
32 Wang, C. et al. Polydopamine—Mediated Surface Functionalization of Exosomes. Chem Nano Mat 7, 592-595, doi:10.1002/cnma.202100078 (2021).
33 Kunze, A., Bally, M., Höök, F. & Larson, G. Equilibrium-fluctuation-analysis of single liposome binding events reveals how cholesterol and Ca2+ modulate glycosphingolipid trans-interactions. Scientific Reports 3, 1452, doi:10.1038/srep01452 (2013).
34 Olsson, C. & Swenson, J. The role of disaccharides for protein-protein interactions—a SANS study. Molecular Physics 117, 3408-3416, doi:10.1080/00268976.2019.1640400 (2019).
35 Kusuma, G. D. et al. To Protect and to Preserve: Novel Preservation Strategies for Extracellular Vesicles. Front Pharmacol 9, 1199, doi:10.3389/fphar.2018.01199 (2018).
36 Thomas, B. L. et al. WNT3A-loaded exosomes enable cartilage repair. J Extracell Vesicles 10, e12088, doi:10.1002/jev2.12088 (2021).
37 Majda, D. et al. New approach for determining cartilage pore size distribution: NaCl-thermoporometry. Microporous and Mesoporous Materials 241, 238-245, doi:https://doi.org/10.1016/j.micromeso.2017.01.005 (2017).
38 El Andaloussi, S., Mäger, I., Breakefield, X. O. & Wood, M. J. A. Extracellular vesicles: biology and emerging therapeutic opportunities. Nature Reviews Drug Discovery 12, 347-357, doi:10.1038/nrd3978 (2013).
39 Tamura, R., Uemoto, S. & Tabata, Y. Augmented liver targeting of exosomes by surface modification with cationized pullulan. Acta Biomaterialia 57, 274-284, doi:https://doi.org/10.1016/j.actbio.2017.05.013 (2017).
40 Cheng, Y., Zeng, Q., Han, Q. & Xia, W. Effect of pH, temperature and freezing-thawing on quantity changes and cellular uptake of exosomes. Protein & Cell 10, 295-299, doi:10.1007/s13238-018-0529-4 (2019).
41 Kusuma, G. D. et al. To Protect and to Preserve: Novel Preservation Strategies for Extracellular Vesicles. Frontiers in Pharmacology 9, doi:10.3389/fphar.2018.01199 (2018).
42 Kreilgaard, L. et al. Effect of Tween 20 on freeze-thawing- and agitation-induced aggregation of recombinant human factor XIII. J Pharm Sci 87, 1597-1603, doi:10.1021/js980126i (1998).
43 Tao, S. C. et al. Exosomes derived from miR-140-5p-overexpressing human synovial mesenchymal stem cells enhance cartilage tissue regeneration and prevent osteoarthritis of the knee in a rat model. Theranostics 7, 180-195, doi:10.7150/thno.17133 (2017).
44 Xu, X. et al. Exosome-mediated delivery of kartogenin for chondrogenesis of synovial fluid-derived mesenchymal stem cells and cartilage regeneration. Biomaterials 269, 120539, doi:https://doi.org/10.1016/j.biomaterials.2020.120539 (2021).
45 Liang, Y. et al. Chondrocyte-Targeted MicroRNA Delivery by Engineered Exosomes toward a Cell-Free Osteoarthritis Therapy. ACS Applied Materials & Interfaces 12, 36938-36947, doi:10.1021/acsami.0c10458 (2020).
46 Wilson, J. T., Krishnamurthy, V. R., Cui, W., Qu, Z. & Chaikof, E. L. Noncovalent Cell Surface Engineering with Cationic Graft Copolymers. Journal of the American Chemical Society 131, 18228-18229, doi:10.1021/ja908887v (2009).
47 Kellam, B., De Bank, P. A. & Shakesheff, K. M. Chemical modification of mammalian cell surfaces. Chemical Society Reviews 32, 327-337, doi:10.1039/B211643J (2003).
48 Richter, M., Vader, P. & Fuhrmann, G. Approaches to surface engineering of extracellular vesicles. Advanced Drug Delivery Reviews 173, 416-426, doi:https://doi.org/10.1016/j.addr.2021.03.020 (2021).
49 Kumariya, R., Sood, S. K., Rajput, Y. S., Saini, N. & Garsa, A. K. Increased membrane surface positive charge and altered membrane fluidity leads to cationic antimicrobial peptide resistance in Enterococcus faecalis. Biochimica et Biophysica Acta (BBA)—Biomembranes 1848, 1367-1375, doi:https://doi.org/10.1016/j.bbamem.2015.03.007 (2015).
50 Ojcius, D. M. & Young, J. D. Cytolytic pore-forming proteins and peptides: is there a common structural motif? Trends Biochem Sci 16, 225-229, doi:10.1016/0968-0004(91)90090-i (1991).
51 Warren, M. R., Vedadghavami, A., Bhagavatula, S. & Bajpayee, A. G. Effects of polycationic drug carriers on the electromechanical and swelling properties of cartilage. Biophys J, doi:10.1016/j.bpj.2022.06.024 (2022).
52 Warren, M. R. & Bajpayee, A. G. Modeling Electrostatic Charge Shielding Induced by Cationic Drug Carriers in Articular Cartilage Using Donnan Osmotic Theory. Bioelectricity, doi:10.1089/bioe.2021.0026 (2021).
53 Young, C. C., Vedadghavami, A. & Bajpayee, A. G. Bioelectricity for Drug Delivery: The Promise of Cationic Therapeutics. Bioelectricity 2, 68-81, doi:10.1089/bioe.2020.0012 (2020).
54 Banks, W. A. et al. Transport of Extracellular Vesicles across the Blood-Brain Barrier: Brain Pharmacokinetics and Effects of Inflammation. Int J Mol Sci 21, doi:10.3390/ijms21124407 (2020).
55 TUMA, P. L. & HUBBARD, A. L. Transcytosis: Crossing Cellular Barriers. Physiological Reviews 83, 871-932, doi:10.1152/physrev.00001.2003 (2003).
56 Sarah E. Headland; Hefin R. Jones; Lucy V. Norling, A. K., Patricia R. Souza, Elisa Corsiero, C. D. G., Alessandra Nerviani, Francesco Dell'Accio, & Costantino Pitzalis, S. M. O., Lily Y. Jan, Mauro Perretti. Neutrophil-derived microvesicles enter cartilage and protect the joint in inflammatory arthritis. Science Translational Medicine. Science Translational Medicine 7 (2015).
57 Grodzinsky, A. J. & Frank, E. H. Fields, forces, and flows in biological systems. (Garland Science, 2011).
58 Li, L. D. et al. Spatial configuration and composition of charge modulates transport into a mucin hydrogel barrier. Biophys J 105, 1357-1365, doi:10.1016/j.bpj.2013.07.050 (2013).
59 Uzieliene, I., Kalvaityte, U., Bernotiene, E. & Mobasheri, A. Non-viral Gene Therapy for Osteoarthritis. Frontiers in Bioengineering and Biotechnology 8, doi:10.3389/fbioe.2020.618399 (2021).
60 Kim, S. H. et al. Exosomes derived from IL-10-treated dendritic cells can suppress inflammation and collagen-induced arthritis. J Immunol 174, 6440-6448, doi:10.4049/jimmunol.174.10.6440 (2005).
61 Qian, X. et al. Immunosuppressive Effects of Mesenchymal Stem Cells-derived Exosomes. Stem Cell Reviews and Reports 17, 411-427, doi:10.1007/s12015-020-10040-7 (2021).
62 Munagala, R., Aqil, F., Jeyabalan, J. & Gupta, R. C. Bovine milk-derived exosomes for drug delivery. Cancer Letters 371, 48-61, doi:https://doi.org/10.1016/j.canlet.2015.10.020 (2016).
63 Tian, Y. et al. Reduction of choroidal neovascularization via cleavable VEGF antibodies conjugated to exosomes derived from regulatory T cells. Nature Biomedical Engineering 5, 968-982, doi:10.1038/s41551-021-00764-3 (2021).
64 Kamerkar, S. et al. Exosomes facilitate therapeutic targeting of oncogenic KRAS in pancreatic cancer. Nature 546, 498-503, doi:10.1038/nature22341 (2017).
65 Momen-Heravi, F., Bala, S., Bukong, T. & Szabo, G. Exosome-mediated delivery of functionally active miRNA-155 inhibitor to macrophages. Nanomedicine 10, 1517-1527, doi:10.1016/j.nano.2014.03.014 (2014).
66 You, Y. et al. Intradermally delivered mRNA-encapsulating extracellular vesicles for collagen-replacement therapy. Nat Biomed Eng, doi:10.1038/s41551-022-00989-w (2023).
67 Jung, M. K. & Mun, J. Y. Sample Preparation and Imaging of Exosomes by Transmission Electron Microscopy. J Vis Exp, doi:10.3791/56482 (2018).
68 Vedadghavami, A., Mehta, S. & Bajpayee, A. G. Characterization of Intra-Cartilage Transport Properties of Cationic Peptide Carriers. J Vis Erp, e61340, doi:10.3791/61340 (2020).
69 Mehta, S., Akhtar, S., Porter, R. M., Onnerfjord, P. & Bajpayee, A. G. Interleukin-1 receptor antagonist (IL-1Ra) is more effective in suppressing cytokine-induced catabolism in cartilage-synovium co-culture than in cartilage monoculture. Arthritis Res Ther 21, 238, doi:10.1186/s13075-019-2003-y (2019).
70 Mehta, S. et al. Resveratrol and Curcumin Attenuate Ex Vivo Sugar-Induced Cartilage Glycation, Stiffening, Senescence, and Degeneration. Cartilage 13, 1214S-1228S, doi:10.1177/1947603520988768 (2021).
71 Scharstuhl, A., van Beuningen, H. M., Vitters, E. L., van der Kraan, P. M. & van den Berg, W. B. Loss of transforming growth factor counteraction on interleukin 1 mediated effects in cartilage of old mice. Annals of the Rheumatic Diseases 61, 1095-1098, doi:10.1136/ard.61.12.1095 (2002).
72 Torzilli, P. A., Bhargava, M., Park, S. & Chen, C. T. Mechanical load inhibits IL-1 induced matrix degradation in articular cartilage. Osteoarthritis Cartilage 18, 97-105, doi:10.1016/j.joca.2009.07.012 (2010).
73 Bajpayee, A. G., Scheu, M., Grodzinsky, A. J. & Porter, R. M. Electrostatic interactions enable rapid penetration, enhanced uptake and retention of intra-articular injected avidin in rat knee joints. J Orthop Res 32, 1044-1051, doi:10.1002/jor.22630 (2014).

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below.

The full scope of the invention should be determined by reference to the claims, along with their full scope of equivalents, and the specification, along with such variations.

We claim:

1. A modified exosome complex, comprising:
(i) an exosome;
(ii) a linking moiety;
(iii) a polypeptide residue or a protein residue;
wherein the exosome comprises a lipid bilayer;
the linking moiety is linked to the lipid bilayer via non-covalent interactions; and
the protein residue or polypeptide residue is covalently linked to the linking moiety.

2. The complex of claim 1, wherein the linking moiety comprises a polymeric moiety.

3. The complex of claim 1 or 2, wherein the linking moiety comprises polyethylene glycol (PEG).

4. The complex of any one of claims 1-3, wherein the linking moiety comprises a lipid moiety.

5. The complex of any one of claims 1-4, wherein the linking moiety comprises 1,2-distearoyl-sn-glycero-3-phosphoethanolamine (DSPE).

6. The complex of any one of claims 1-5, wherein the linking moiety comprises DSPE-PEG.

7. The complex of any one of claims 1-6, wherein the non-covalent interaction is hydrophobic partitioning.

8. The complex of any one of claims 1-7, wherein the linking moiety comprises a triazole moiety.

9. The complex of any one of claims 1-8, wherein the linking moiety comprises a residue of dibenzocyclooctene.

10. The complex of any one of claims 1-7, wherein the linking moiety comprises DSPE-PEG-Biotin.

11. The complex of claim 10, wherein the complex comprises an exosome, a linking moiety, and a protein residue.

12. The complex of claim 11, wherein the protein residue is avidin.

13. The complex of any one of claims 1-9 wherein the complex comprises an exosome, a linking moiety, and a polypeptide residue.

14. The complex of claim 13, wherein the polypeptide residue comprises 2 to 40 amino acid residues, and the net charge of the polypeptide is +7 to +20.

15. The complex of claim 13 or 14, wherein the polypeptide comprises at least one arginine residue, lysine residue, or other positively charged amino acid residue.

16. The complex of any one of claims 13-15, wherein the polypeptide comprises at least one arginine residue or lysine residue.

17. The complex of any one of claims 13-16, wherein the polypeptide comprises (i) a plurality of arginine residues, and a plurality of alanine residues, or (ii) a plurality of arginine residues, and a plurality of asparagine residues, or (iii) a plurality of arginine residues, and a mixture of alanine and asparagine residues; or (iv) a plurality of lysine residues, and a plurality of alanine residues; or (v) a plurality of lysine residues, and a plurality of asparagine residues; or (vi) a plurality of lysine residues, and a mixture of alanine and asparagine residues; or (vii) a plurality of arginine residues.

18. The complex of any one of claims 13-17, wherein the net charge of the polypeptide residue is +7 to +14.

19. The complex of any one of claims 13-18, wherein the net charge of the polypeptide is +8.

20. The complex of any one of claims 13-19, wherein the net charge of the polypeptide is +14.

21. The complex of any one of claims 13-20, wherein the polypeptide residue is selected from the group consisting of: AKAKAKAKAKAKAKANANAN;
RRAAAARRAAAARRAAAARR;
RRRRAARRRAARRRAARRRR;
(ARRRAARA)₄;
RRRRRRRRRRRRRRRRRRRR;
and
RRRR(NNRRR)₃R.

22. The complex of any one of claims 13-21, wherein the polypeptide residue is RRRR(NNRRR)₃R.

23. A method of preparing the modified exosome complex of any one of claims 1-22, comprising:
(a) combining a linking moiety and an exosome comprising a lipid bilayer, thereby associating the linking moiety with the lipid bilayer of the exosome via non-covalent interactions;
(b) combining a polypeptide residue or protein residue with a buffer solution to neutralize the charge of the polypeptide residue or protein residue;
(c) combining the charge-neutralized polypeptide residue or protein residue with the linking moiety associated with the lipid bilayer of the exosome, thereby forming a covalent linkage between the charge-neutralized polypeptide residue or protein residue and the linking moiety; and
(d) bringing the buffer solution to physiological pH and salinity.

24. A method of encapsulating RNA into an exosome comprising:
(a) combining lipofectamine and a solution comprising RNA, thereby forming a first mixture;
(b) combining the first mixture and an exosome, thereby forming a second mixture; and
(c) combining the second mixture with RNase.

25. The method of claim 24, wherein the exosome is the modified exosome complex of any one of claims 1-22.

26. The method of claim 24, wherein the exosome is a native exosome.

27. A method of delivering a therapeutic agent to a negatively charged tissue, comprising administering to a subject in need thereof a therapeutically effective amount of a composition;
wherein the composition comprises the modified exosome complex of any one of claims 1-22; and a therapeutic agent.

28. The method of claim 27, wherein the therapeutic agent is a nucleic acid, a protein, or a small molecule drug.

29. The method of claim 28, wherein the therapeutic agent is a nucleic acid.

30. The method of claim 29, wherein the nucleic acid comprises RNA or a plasmid vector.

31. The method of claim 30, wherein the RNA is an mRNA.

32. The method of claim 30, wherein the RNA is an siRNA.

33. The method of claim 30 or 31, wherein the RNA is eGFP mRNA.

34. The method of any one of claims 27-33, wherein administering the composition comprises intra-articular injection.

35. The method of any one of claims 27-33, wherein administering the composition comprises oral administration.

36. The method of any one of claims 27-33, wherein administering the composition comprises transmucosal administration.

37. The method of any one of claims 27-36, wherein the negatively charged tissue is selected from the group consisting of cartilage, meniscus, tendons, ligaments, fracture callus, retina, intervertebral disc, mucosal membrane, and malignant tissue.

38. The method of any one of claims 27-37, wherein the negatively charged tissue is cartilage.

39. The method of any one of claims 27-37, wherein the negatively charged tissue is mucosal membrane.

40. A method of treating a joint disease, comprising administering to a subject in need thereof a therapeutically effective amount of a composition;
wherein the composition comprises the modified exosome complex of any one of claims 1-22; and a therapeutic agent.

41. The method of claim 40, wherein the joint disease is selected from the group consisting of rheumatoid arthritis, spondyloarthritis, juvenile idiopathic arthritis, lupus, gout, bursitis, and osteoarthritis.

42. The method of claims 40 or 41, wherein the joint disease is osteoarthritis.

43. The method of any one of claims 40-42, wherein the therapeutic agent is a nucleic acid, a protein, or a small molecule drug.

44. The method of claim 43, wherein the therapeutic agent is a nucleic acid.

45. The method of claim 44, wherein the nucleic acid comprises RNA or a plasmid vector.

46. The method of claim 45, wherein the RNA is an mRNA.

47. The method of claim 45, wherein the RNA is an siRNA.

48. The method of claim 45 or 46, wherein the RNA is eGFP mRNA.

49. A method of preparing the modified exosome complex of any one of claims 1-22, comprising:
(i) combining a polypeptide residue or protein residue with a linking moiety, thereby forming a covalent linkage between the polypeptide residue or protein residue and the linking moiety;
(j) combining the polypeptide residue or protein residue with a buffer solution to neutralize the charge of the polypeptide residue or protein residue;
(k) combining the linking moiety and an exosome comprising a lipid bilayer, thereby associating the linking moiety with the lipid bilayer of the exosome via non-covalent interactions;
(l) bringing the buffer solution to physiological pH and salinity.
